(19) United States
(12) Patent Application Publication
(10) Pub. No.: US 2011/0086891 A1
(43) Pub. Date: Apr. 14, 2011

(54) Solid Forms of 2-(2,4-Difluorophenyl)-6-(1-(2,6-Difluorophenyl)Ureido)Nicotinamide)

(75) Inventors: Michael Hurrey, Maynard, MA (US); Dimitar Alargov, Brighton, MA (US); Steven C. Johnston, Bolton, MA (US); Stefanie Roeper, Cambridge, MA (US); John R. Snoonian, Bolton, MA (US); Brett A. Cowans, West Lafayette, IN (US); Petinka Vlahova, West Lafayette, IN (US); Alexander Eberlin, Cambridge (GB); Mark Eddleston, Cambridge (GB); Christopher Frampton, Suffolk (GB)

(73) Assignee: Vertex Pharmaceuticals Incorporated, Cambridge, MA (US)

(21) Appl. No.: 12/705,105

(22) Filed: Feb. 12, 2010

Related U.S. Application Data
(60) Provisional application No. 61/152,648, filed on Feb. 13, 2009.
(60) Provisional application No. 61/157,839, filed on Mar. 5, 2009.

(51) Int. Cl. A61K 31/465; C07D 213/82; A61P 29/00; A61P 19/10

(52) U.S. Cl. 514/353; 546/306

(57) ABSTRACT

This invention relates to solid forms of 2-(2,4-difluorophenyl)-6-(1-(2,6-difluorophenyl)ureido)nicotinamide and pharmaceutical compositions thereof, and methods and uses therewith.

### CROSS-REFERENCE TO RELATED APPLICATIONS

This application claims priority to U.S. provisional application Ser. Nos. 61/152,648 and 61/157,839, which were filed on Feb. 13, 2009, and Mar. 5, 2009, respectively. The entire contents of both provisional applications are incorporated herein in their entirety.

### TECHNICAL FIELD OF THE INVENTION

This invention relates to solid forms of 2-(2,4-difluorophenyl)-6-(1-(2,6-difluorophenyl)ureido)nicotinamide and pharmaceutical compositions thereof, and methods and uses therewith.

### BACKGROUND OF THE INVENTION

Protein kinases are involved in various cellular responses to extracellular signals. Recently, a family of mitogen-activated protein kinases (MAPK) has been discovered. Members of this family are Ser/Thr kinases that activate their substrates by phosphorylation [B. Stein et al., Ann. Rep. Med. Chem., 31, pp. 289-98 (1996)]. MAPKs are themselves activated by a variety of signals including growth factors, cytokines, UV radiation, and stress-inducing agents.

One particularly interesting MAPK is p38. p38, also known as cytokine suppressive anti-inflammatory drug binding protein (CSBP) and RK, was isolated from murine pre-B cells that were transfected with the lipopolysaccharide (LPS) receptor, CD14, and induced with LPS. p38 has since been isolated and sequenced, as has the cDNA encoding it in humans and mice. Activation of p38 has been observed in cells stimulated by stress, such as treatment with bacterial lipopolysaccharides (LPS, also called endoxin), UV, anisomycin, or osmotic shock, and by cytokines, such as IL-1 and TNF.

Inhibition of p38 kinase leads to a blockade on the production of both IL-1 beta and TNF alpha. IL-1 and TNF stimulate the production of other proinflammatory cytokines such as IL-6 and IL-8 and have been implicated in acute and chronic inflammatory diseases and in post-menopausal osteoporosis [R. B. Kimble et al., Endocrinol., 136, pp. 3054-61 (1995)].

Based upon this finding, it is believed that p38, along with other MAPKs, have a role in mediating cellular response to inflammatory stimuli, such as leukocyte accumulation, macrophage/monocyte activation, tissue resorption, fever, acute phase responses and neutrophilia. In addition, MAPKs, such as p38, have been implicated in cancer, thrombin-induced platelet aggregation, immunodeficiency disorders, autoimmune disease, cell death, allergies, asthma, osteoporosis and neurodegenerative diseases. Inhibitors of p38 have also been implicated in the area of pain management through inhibition of prostaglandin endoperoxide synthase-2 induction. Other diseases associated with IL-1, IL-6, IL-8 or TNF over-production were set forth in WO 96/21654.

2-(2,4-difluorophenyl)-6-(1-(2,6 difluorophenyl)ureido)nicotinamide (Compound I) having the structure depicted below, has demonstrated efficacy for the treatment of a variety of diseases, including inflammatory diseases. Compound I is described in WO 2004/72038, which was published on Aug. 26, 2004.

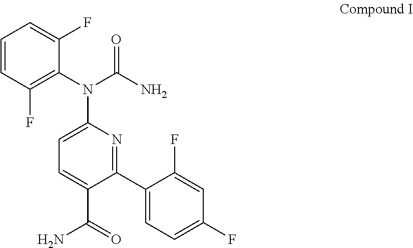

### SUMMARY OF THE INVENTION

The present invention provides a description of solid forms of Compound I. The properties of a solid relevant to its efficacy as a drug can be dependent on the form of the solid. For example, in a drug substance, variation in the solid form can lead to differences in properties such as melting point, dissolution rate, oral absorption, bioavailability, toxicology results and even clinical trial results. In some embodiments the solid forms of Compound I are neat forms. In other embodiments, the solid forms of Compound I are co-forms, for example salts, solvates, co-crystals and hydrates.

Isotopically-labeled forms of Compound I wherein one or more atoms are replaced by an atom having an atomic mass or mass number different from the atomic mass or mass number usually found in nature are also included herein. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, and ¹⁷O, Such radio-labeled and stable-isotopically labeled compounds are useful, for example, as research or diagnostic tools.

The present invention also provides strategies for the control of solid forms that arise during the manufacture of Compound I.

In another aspect, the solid forms of Compound I described herein and their pharmaceutically acceptable compositions are useful in methods for treating or lessening the symptoms of a variety of diseases, which include acute and chronic inflammatory diseases, cancer, autoimmune disease, immunodeficiency disorders, destructive bone disorders (e.g., post-menopausal osteoporosis), proliferative disorders, infectious diseases, viral diseases, allergies, asthma, burns and neurodegenerative diseases. These solid forms and compositions are also useful in methods for preventing cell death and hyperplasia and therefore might be used to treat or prevent reperfusion/ischemia in stroke, heart attacks and organ hypoxia. These solid forms and compositions are also useful in methods for preventing thrombin-induced platelet aggregation.

In another aspect, the solid forms of Compound I described herein and their pharmaceutically acceptable compositions are also useful for the study of p38 kinases in biological and pathological phenomena, the study of intracellular signal transduction pathways mediated by such kinases and the comparative evaluation of new kinase inhibitors.

### DETAILED DESCRIPTION OF THE INVENTION

### Definitions and General Terminology

As used herein, the term “crystalline” refers to a solid that has a specific arrangement and/or conformation of the molecules in the crystal lattice.

As used herein the term “amorphous” refers to solid forms that consist of disordered arrangements of molecules and do not possess a distinguishable crystal lattice.

As used herein, the term “solvate” refers to a crystalline solid adduct containing either stoichiometric or nonstoichiometric amounts of a solvent incorporated within the crystal structure. If the incorporated solvent is water, such adduct is referred to as a “hydrate”.

As used herein, the term “pharmaceutically acceptable salt” refers to those salts which are, within the scope of sound medical judgment, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

The term “chemically stable”, as used herein, means that the solid form of Compound I does not decompose into one or more different chemical compounds when subjected to specified conditions, e.g., 40° C./75% relative humidity (RH), for a specific period of time. e.g. 1 day, 2 days, 3 days, 1 week, 2 weeks, or longer. In some embodiments, less than 25% of the solid form of Compound I decomposes, in some embodiments, less than about 20%, less than about 15%, less than about 10%, less than about 5%, less than about 3%, less than about 1%, less than about 0.5% of the form of Compound I decomposes under the conditions specified. In some embodiments, no detectable amount of the solid form of Compound I decomposes.

The term “physically stable”, as used herein, means that the solid form of Compound I does not change into one or more different physical forms of Compound I (e.g. different solid forms as measured by XRPD, DSC, etc.) when subjected to specific conditions, e.g., 40° C./75% relative humidity, for a specific period of time. e.g. 1 day, 2 days, 3 days, 1 week, 2 weeks, or longer. In some embodiments, less than 25% of the solid form of Compound I changes into one or more different physical forms when subjected to specified conditions. In some embodiments, less than about 20%, less than about 15%, less than about 10%, less than about 5%, less than about 3%, less than about 1%, less than about 0.5% of the solid form of Compound I changes into one or more different physical forms of Compound I when subjected to specified conditions. In some embodiments, no detectable amount of the solid form of Compound I changes into one or more physically different solid forms of Compound I.

The term “substantially free” (as in the phrase “substantially free of form X”) when referring to a designated solid form of Compound I (e.g., an amorphous or crystalline form described herein) means that there is less than 20% (by weight) of the designated form(s) or co-form(s) (e.g., a crystalline or amorphous form of Compound I) present, more preferably, there is less than 10% (by weight) of the designated form(s) present, more preferably, there is less than 5% (by weight) of the designated form(s) present, and most preferably, there is less than 1% (by weight) of the designated form(s) present.

The term “substantially pure” when referring to a designated solid form of Compound I (e.g., an amorphous or crystalline solid form described herein) means that the designated solid form contains less than 20% (by weight) of residual components such as alternate polymorphic or isomorphic crystalline form(s) or co-form(s) of Compound I. It is preferred that a substantially pure solid form of Compound I contains less than 10% (by weight) of alternate polymorphic or isomorphic crystalline forms of Compound I, more preferably less than 5% (by weight) of alternate polymorphic or isomorphic crystalline forms of Compound I, and most preferably less than 1% (by weight) of alternate polymorphic or isomorphic crystalline forms of Compound I.

This application often refers to evaluating a “chemical or physical” parameter disclosed herein. Such parameters can be substituted with other chemical or physical parameters which though not disclosed herein are essentially similar in terms of identifying the form and well known to one skilled in the art.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts an exemplary XRPD trace for Form C.

FIG. 2 depicts an exemplary ¹H NMR spectrum for Form C.

FIG. 3 depicts an exemplary FT-IR spectrum for Form C.

FIG. 4 depicts an exemplary DSC trace for Form C.

FIG. 5 depicts an exemplary TGA trace for Form C.

FIG. 6 depicts the characteristic X-Ray diffraction packing diagram for Form C.

FIG. 7 depicts an scheme of the crystal structure for Form C as seen by single crystal X-Ray crystallography.

FIG. 8 depicts an exemplary GVS trace for Form C.

FIG. 9 depicts the results of stability studies for Form C as seen by XRPD, in which the before and after spectra are similar.

FIG. 10 depicts a characteristic HPLC for pure Form C.

FIG. 11 depicts an exemplary XRPD trace for Form F.

FIG. 12 depicts an exemplary ¹H NMR spectrum for Form F.

FIG. 13 depicts an exemplary FT-IR spectrum for Form F.

FIG. 14 depicts an exemplary DSC trace for Form F.

FIG. 15 depicts an exemplary TGA trace for Form F.

FIG. 16 depicts an exemplary GVS trace for Form F.

FIG. 17 depicts an exemplary XRPD trace for Form G.

FIG. 18 depicts an exemplary ¹H NMR spectrum for Form G.

FIG. 19 depicts an exemplary FT-IR spectrum for Form G.

FIG. 20 depicts an exemplary DSC trace for Form G.

FIG. 21 depicts an exemplary TGA trace for Form G.

FIG. 22 depicts an exemplary GVS trace for Form G.

FIG. 23 depicts the results of stability studies for Form G as seen by XRPD in which the before and after spectra are similar.

FIG. 24 depicts an exemplary XRPD trace for Form A.

FIG. 25 depicts an exemplary DSC trace for Form A.

FIG. 26 depicts an exemplary TGA trace for Form A.

FIG. 27 depicts an exemplary FT-IR spectrum for Form A.

FIG. 28 depicts the results of stability studies for Form A as seen by XRPD, in which the before and after spectra illustrate formation of Form C.

FIG. 29 depicts an exemplary XRPD trace for Form Q.

FIG. 30 depicts an exemplary XRPD trace for Form P.

FIG. 31 depicts an exemplary ¹H NMR spectrum for Form P.

FIG. 32 depicts an exemplary TGA trace for Form P.

FIG. 33 depicts an exemplary DSC trace for Form P.

FIG. 34 depicts an exemplary FT-IR spectrum for Form P.

FIG. 35 depicts the results of stability studies for Form P as seen by XRPD, in which the before and after spectra illustrate formation of Form C.

FIG. 36 depicts a flow chart which illustrates how to convert various solid forms into other solid forms. The conditions depicted in the figure are as follows:
i. heating above 50° C.
ii. cooling below 50° C.
iii. slurry rt, MeOH.
iv. storage at 4° C., 2-4 wks.
v. slurry at 0° C. in 1:3 H₂O/MeOH for 24 h or slurry in H₂O for 3 days/0° C., then at 25° C. for 3 days.
vi. slurry in MeOH above 50° C.
vii. crystallization method M from MeOH.
viii. slurry in non solvate forming solvent (e.g. 1:1 MeOH/H₂O).
ix. slurry in ethyl acetate/hexanes at −20° C., for 24 h.
x. heat above 130° C.
xi. slurry in ethyl acetate/hexanes at −20° C., for 2 h.
xii. slurry in EtOAc.
xiii. dry at rt.
xiv. heat at 100° C. to desolvate.

### DESCRIPTION OF SOLID FORMS OF COMPOUND I AND METHODS OF CHARACTERIZATION THEREOF

Compound I has been prepared in various solid forms, including three neat crystalline forms (Forms C, F and G), and four solvates, which in turn can appear as solvates or as their corresponding de-solvated solvates (Forms A, O, P and Q). The form identification or ID, chemical name and the co-solvent in the case of solvates, for each of these solid forms are provided in Table I below:

TABLE I
Solid Forms of Compound I

| | | Neat | |
|---|---|---|---|
| Form | | Form | Solvent |
| ID | Chemical Name | (y/n) | (API*:Solvent) |
| A | 2-(2,4-difluorophenyl)-6-(1-(2,6- | n | MeOH |
| | difluorophenyl)ureido)nicotinamide•MeOH | | (1:1) |
| C | 2-(2,4-difluorophenyl)-6-(1-(2,6- | y | N/A |
| | difluorophenyl)ureido)nicotinamide | | |
| F | 2-(2,4-difluorophenyl)-6-(1-(2,6- | y | N/A |
| | difluorophenyl)ureido)nicotinamide | | |
| G | 2-(2,4-difluorophenyl)-6-(1-(2,6- | y | N/A |
| | difluorophenyl)ureido)nicotinamide | | |
| O | 2-(2,4-difluorophenyl)-6-(1-(2,6- | n | Ethyl Acetate |
| | difluorophenyl)ureido)nicotinamide•Ethyl | | (1:1) |
| | Acetate | | |
| P | 2-(2,4-difluorophenyl)-6-(1-(2,6- | n | MeOH |
| | difluorophenyl)ureido)nicotinamide•MeOH | | (1:1) |
| Q | 2-(2,4-difluorophenyl)-6-(1-(2,6- | n | water |
| | difluorophenyl)ureido)nicotinamide•H₂O | | (1:1) |

(*API = active pharmaceutical ingredient)

The solid forms of Compound I described in Table I can be made as described herein. FIG. 36 illustrates how to convert various solid forms into other solid forms.

The methods described in FIG. 36 represent exemplary routes for producing solid forms A, C, F, G, O, P, and Q are not meant to be limiting. Other routes not described herein may be useful for producing solid forms A, C, F, G, O, P, and Q. In some instances, solid forms A, P, C, F, O, and G may revert to form Q when slurried in water.

Each of the solid forms outlined above were analyzed using one or more analytical techniques described herein: single crystal X-Ray analysis, X-Ray powder diffraction (XRPD), differential scanning calorimetry (DSC), thermogravimetric analysis (TGA), gravimetric vapor absorption (GVS), ¹H Nuclear Magnetic Resonance (NMR), Fourier-transform IR (FT-IR), temperature gradient IR (TG-IR), stability analysis (e.g chemical and/or physical stability analyses), hygroscopicity, and solubility analysis.

### Preparation and Characterization of Neat Forms of Compound I

### Description of Crystallization Techniques Employed

Slow Evaporation (SE):

A weighed amount of Compound I, Form A was treated with aliquots of the test solvent. Between additions, the mixture was shaken or sonicated. When all the solids were dissolved, as judged by visual inspection, the solution was filtered, and then left under ambient conditions in a vial covered with aluminium foil containing pinholes.

Fast Evaporation (FE):

A weighed amount of Compound I, Form A was treated with aliquots of the test solvent. Between additions, the mixture was shaken or sonicated. When all the solids were dissolved, as judged by visual inspection, the solution was filtered, and then left in an open vial under ambient conditions.

Crash Cool (CC) or Fast Cool (FC):

A weighed amount of Compound I, Form A was treated with aliquots of the test solvent. Between additions, the mixture was shaken or sonicated. The solution was then heated at 60° C. by keeping the mixture on a hot plate. The resulting solution was rapidly filtered into a vial kept on the same hot plate. The heat source was turned off and the vial capped and transferred to a 5° C. freezer to allow for crystallization.

Slow Cool (SC):

A weighed amount of Compound I, Form A was treated with aliquots of the test solvent. Between additions, the mixture was shaken or sonicated. The solution was then heated at 60° C. by keeping the mixture on a hot plate. The resulting solution was rapidly filtered into a vial kept on the same hot plate. The heat source was turned off and the vial capped and kept capped at ambient temperature to allow for crystallization.

Ground (G):

The solid (usually Form A) was ground with a spatula or mortar and pestle for a given amount of time (generally given in seconds).

Slurry:

Slurry experiments were carried out by making saturated solutions containing excess solid (this applies to any of the solid form described herein). The slurries were agitated at ambient temperatures for up to 2 months. The insoluble solids were recovered, either by filtration or decantation and air-dried.

Maturation in a Range of Solvents (M):

100 mg of Compound I, Form C was weighed into a small, screw top vial. The given solvent was added. The vial was then subjected to 3 heat/cool cycles between ambient temperature and 50° C. over a 20 hour period with shaking. An observation was then made as to whether the vial contained a solution or a slurry (i.e. un-dissolved solid). The solution/slurry was then filtered hot through a pre-heated 0.45 mm PFTE filter. The filtered solid was retained and analyzed by XRPD. The filtrate was allowed to cool to room temperature in a capped vial to encourage precipitation. If no precipitation occurred, the vial was stored at 4° C. and then un-capped to allow evaporation. Resulting precipitates were also analyzed by XPRD where appropriate.

### Preparation and Characterization of Form C

Form C is a crystalline form of Compound I and can be prepared from crystalline Form A, the method comprising the steps of
i) slurrying methanol solvate Form A in 20 volumes of a 1:3 methanol:water mixture for 24 hours (a kinetically controlled step that produces Form C and Form Q/G, described below), and
ii) slurrying the resulting mixture in a 1:1 methanol:water mixture to suppress formation of Form Q/G and favor thermodynamically more stable Form C.

In another embodiment, Form C can be obtained by preparing a slurry of Form A in EtOAc for 18 days. In another embodiment, Form C can be obtained by slurrying Form A in toluene for 7 days. In a further embodiment, Form C can be obtained by slurrying Form A in water for 7 days. In a further embodiment, Form C can be obtained by slurrying Form A in i-PrOH:H₂O (8:2) for 4 days. In another embodiment, Form C can be obtained by slurrying Form A in acetonitrile/H₂O (2:8) for 7 days. In a further embodiment, Form C can be obtained by slurrying Form A in MeOH:H₂O (2:8) for 7 days. In yet another embodiment, Form C can be obtained by slurrying Form A in acetone:H₂O (2:8) for 7 days.

In another embodiment, method FE described above and EtOAc as the test solvent can be used to prepare Form C starting from Form A.

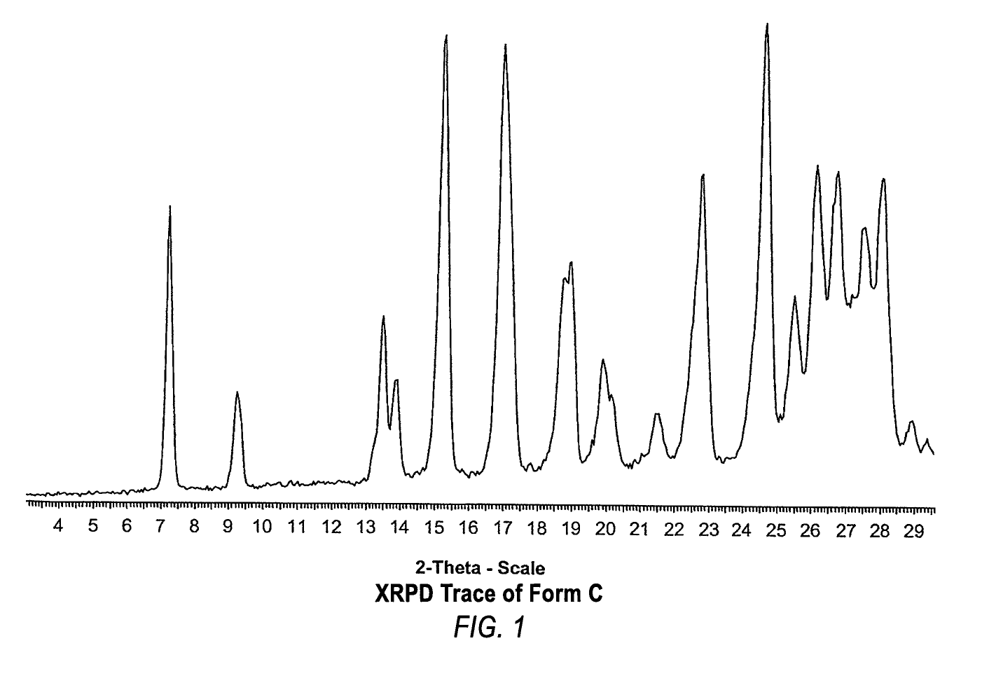

Form C can be characterized by the X-Ray powder diffraction pattern depicted in FIG. 1. Representative peaks as observed in the XRPD spectrum are provided in Table II below:

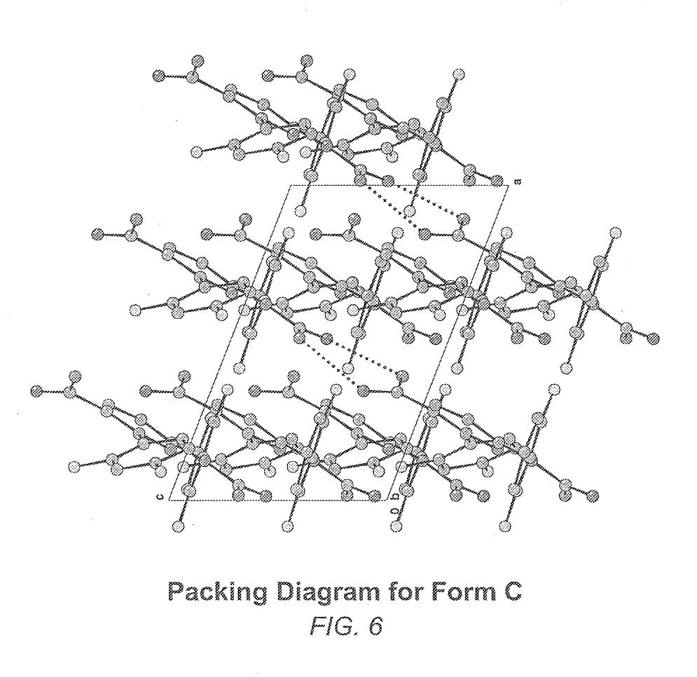

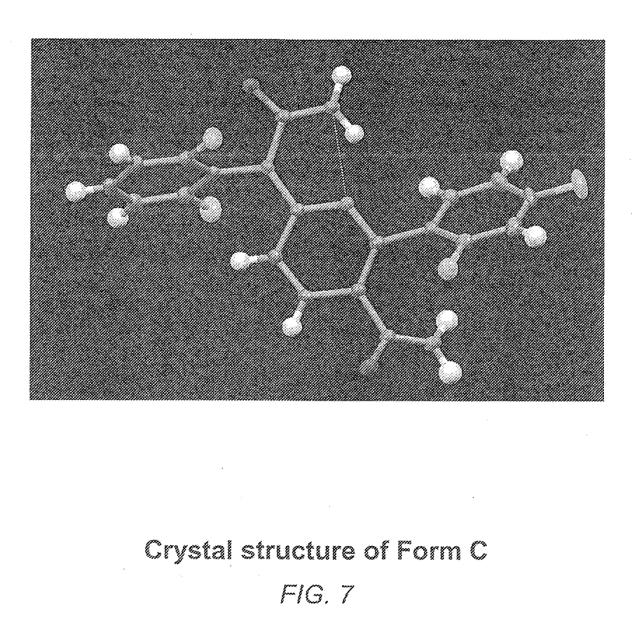

A single crystal X-Ray has been obtained from a crystal of Form C obtained by crystallization of Form A from EtOAc (by using method SE). A schematic of the crystal packing is depicted in FIG. 6. As revealed by single crystal X-Ray crystallography, Form C has a space group C_{c} having the following unit cell dimensions:
a=10.9241 Å, b=24.2039 Å, c=7.0124 Å
α=90°, β=111.0685°, γ=90°
δ_{calc} (g/cm³)=1.552

TABLE II
Representative XRPD peaks for Form C

| Angle 2-θ (°) | d value (Å) | Intensity (%) |
|---|---|---|
| 7.4 | 11.85 | 100 |
| 9.5 | 9.29 | 23.1 |
| 13.7 | 6.45 | 22.3 |
| 14.1 | 6.28 | 18.2 |
| 15.5 | 5.7 | 51 |
| 17.2 | 5.14 | 35.5 |
| 19.2 | 4.62 | 21.6 |
| 22.9 | 3.87 | 20.8 |
| 24.8 | 3.58 | 31.6 |
| 26.3 | 3.38 | 16.2 |
| 26.9 | 3.30 | 17.2 |
| 27.7 | 3.2 | 14.6 |
| 28.3 | 3.15 | 18.7 |

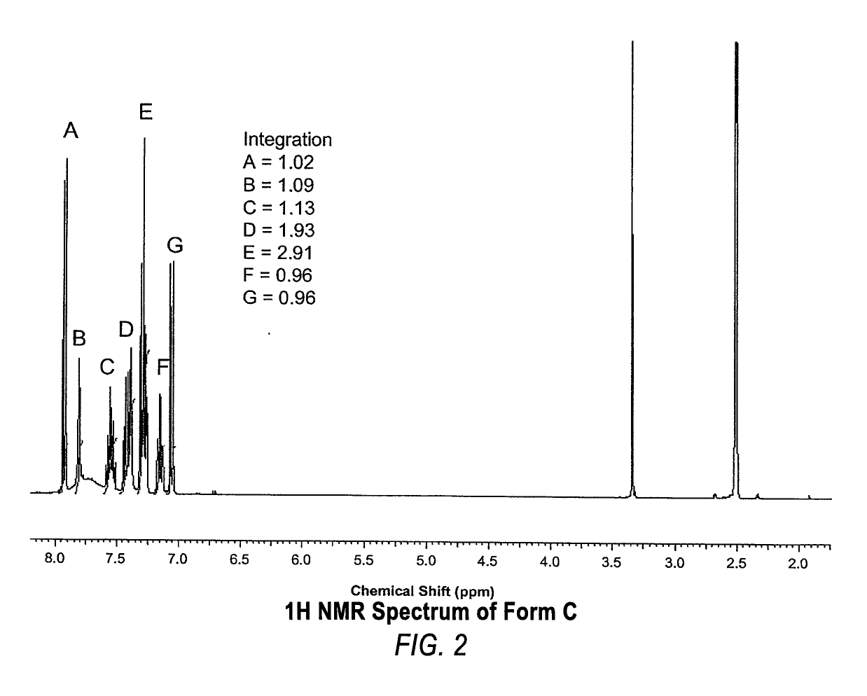

Form C can be characterized by a ¹H NMR spectrum as depicted in FIG. 2. Exemplary peaks include one of more of the following as measured in ppm

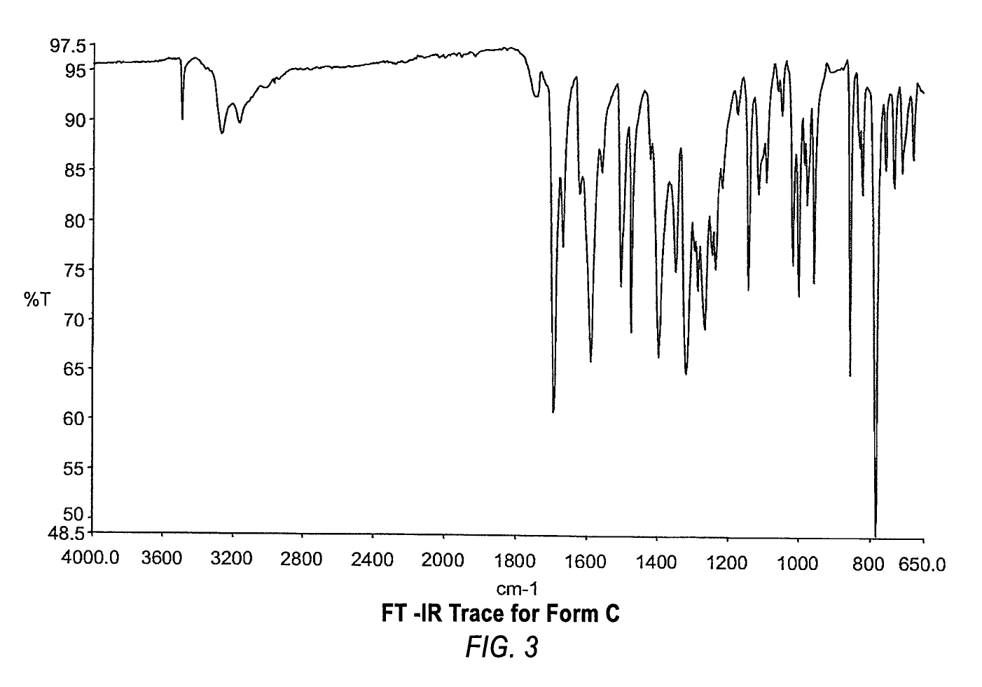

Form C can be characterized by a FT-IR spectrum as depicted in FIG. 3.

Form C can be characterized by an endotherm beginning at 178° C., that plateaus slightly and then peaks at 193° C. as measured by DSC. Further, this endotherm coincides with a 9.5-10.5% weight loss as measured by TGA and attributed to chemical degradation.

Form C displays solubility in water of at least 0.02 mg/mL at 25° C.

Form C remains in substantially the same physical form for at least two weeks at 40° C./75% RH. Further, it displays a negligible weight gain up to 60% Relative Humidity (RH) and a low total weight gain of 0.15% from 0 to 90% RH at T=25° C.

Form C remains chemically stable for at least 2 weeks at 40° C./75% RH.

### Preparation and Characterization of Form F

Form F is a crystalline form of Compound I.

Crystalline Form F can be prepared from Form C, the method comprising the steps of:
i) preparing an ethyl acetate slurry of Form C,
ii) inducing precipitation with cold hexanes for 2 h, and
iii) filtering and drying the resulting solid to furnish Compound I, Form F.

In another embodiment, Form F can be obtained from Form G (described below) upon heating Form G at 120° C. under atmospheric pressure.

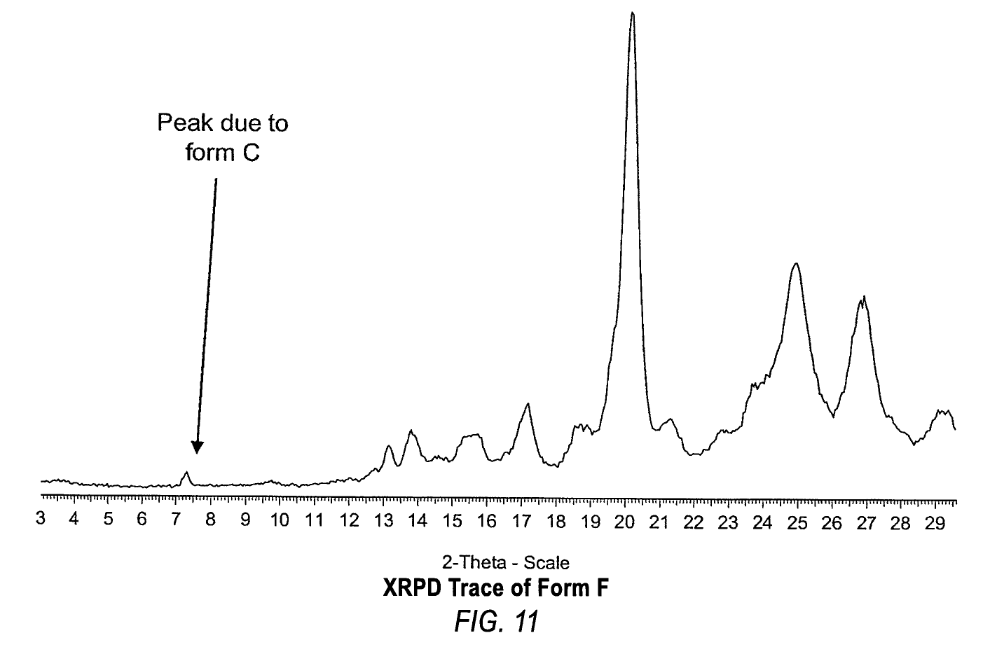

A representative XRPD pattern of Form F is provided in FIG. 11. presentative peaks as observed in the XRPD are provided in Table III below:

TABLE III
Representative XRPD peaks for Form F

| Angle 2-θ (°) | d value (Å) | Intensity (%) |
|---|---|---|
| 14.0 | 6.32 | 78.5 |
| 15.6 | 5.55 | 78.8 |
| 17.3 | 5.11 | 76 |
| 19.1 | 4.64 | 75.5 |
| 20.4 | 4.34 | 100 |
| 23.1 | 3.85 | 73.8 |
| 24.9 | 3.57 | 75.8 |

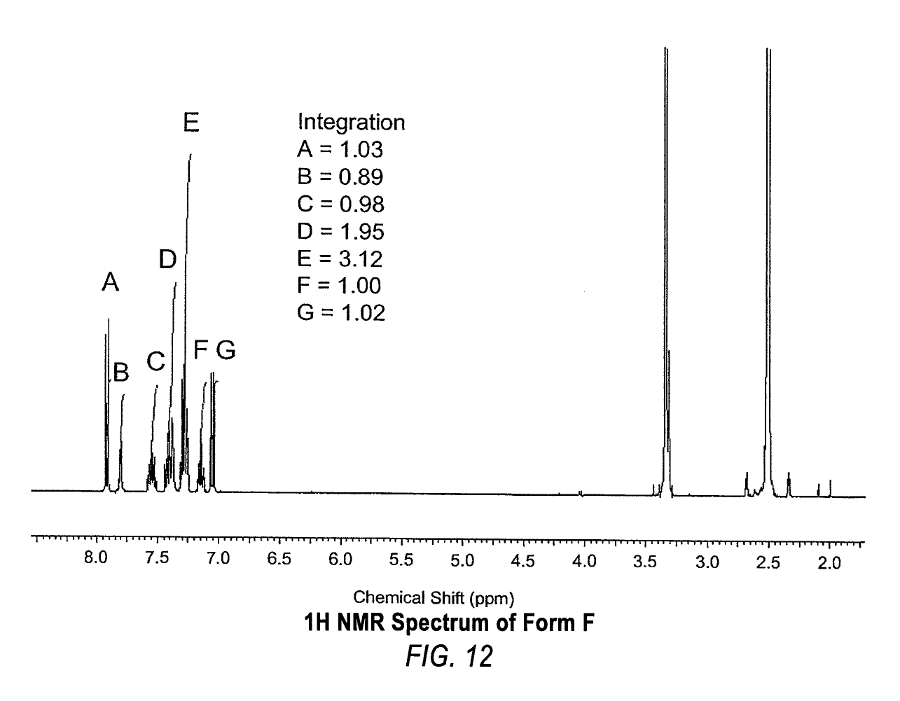

Form F can be characterized by a ¹H NMR spectrum as depicted in FIG. 12.

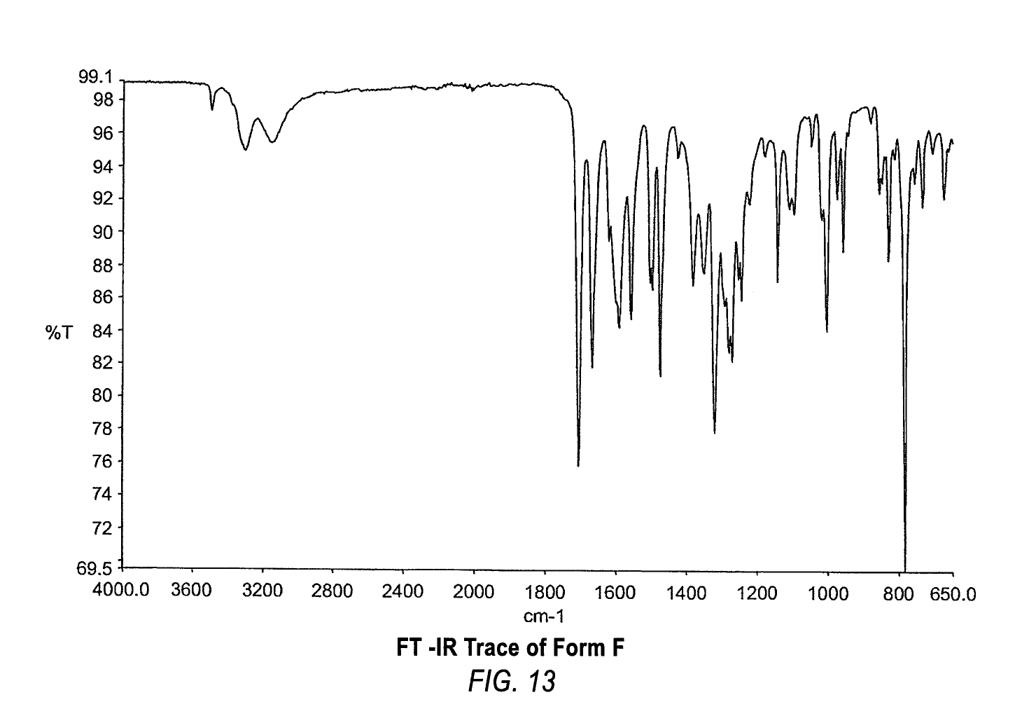

Form F can be characterized by a FT-IR spectrum as depicted in FIG. 13.

Form F is characterized by an endothermal event beginning at 160° C. and peaking at 165° C. as measured by DSC. Further, this thermal event coincides with a 6.8% net weight loss between 130° C. and 180° C. as measured by TGA and attributed to chemical degradation.

Form F displays solubility in water of at least 0.021 mg/mL at 25° C.

Form F remains in substantially the same physical form for at least 2 weeks at 40° C./75% RH. Further, Form F remains chemically stable for at least 2 weeks at 40° C./75% RH.

Form F displays a total weight gain in water of 1% at 40% RH and a maximum of 1.1% at 90% RH as seen by GVS.

### Preparation and Characterization of Form G

Form G is a crystalline form of Compound I. Further, in the presence of water Form G becomes its hydrate, Form Q.

Crystalline Form G can be prepared from crystalline Form C, the method comprising the steps of:
i) preparing an ethyl acetate slurry of Form C,
ii) inducing precipitation with cold hexanes for 24 h, and
iii) filtering and drying the resulting solid to furnish Compound I, Form G.

In another embodiment, Form G can be prepared by slurrying Form A in water at 0° C. for 3 days and then at 25° C. for an additional 3 days followed by drying.

In yet another embodiment, Form G can be obtained by preparing a slurry of Form A in MeOH:H₂O 8:2 for 24 hours.

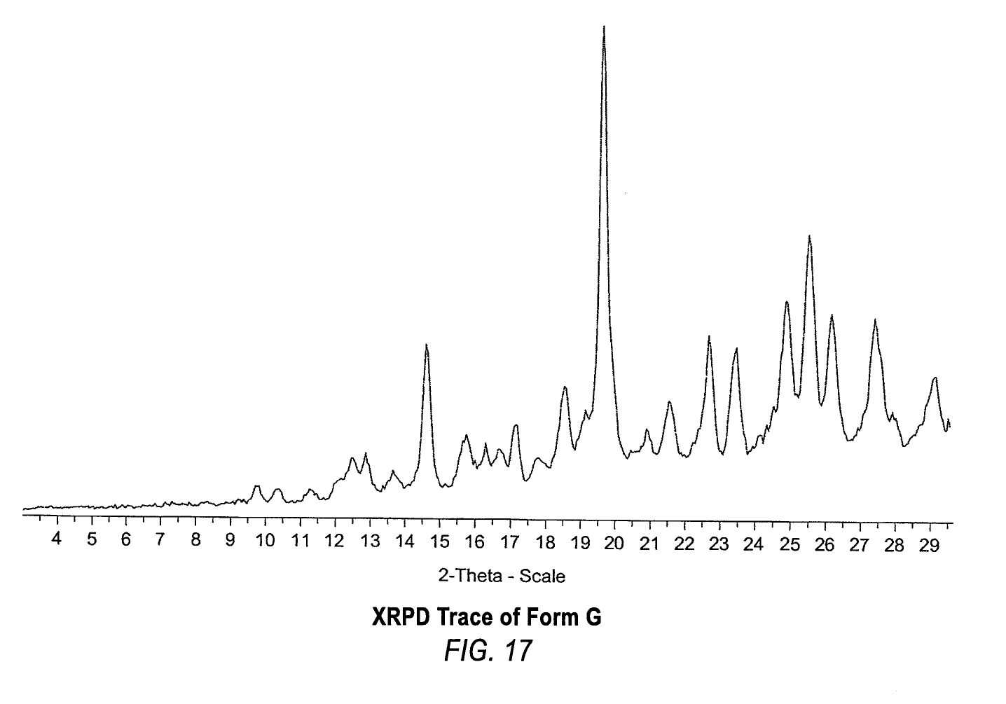

A representative XRPD pattern of Form G is provided in FIG. 17. Representative peaks as observed in the XRPD are provided in Table IV below:

TABLE IV
Representative XRPD peaks for Form G

| Angle 2-θ (°) | d value (Å) | Intensity (%) |
|---|---|---|
| 9.9 | 8.89 | 43.6 |
| 14.8 | 5.97 | 68.9 |
| 17.3 | 5.11 | 45.2 |
| 18.8 | 4.75 | 45.2 |
| 19.8 | 4.48 | 100 |
| 21.7 | 4.08 | 42.7 |
| 22.7 | 3.90 | 45 |
| 23.6 | 3.77 | 42.5 |
| 27.7 | 3.22 | 46.7 |

Form G can be characterized by a ¹H NMR spectrum as depicted in

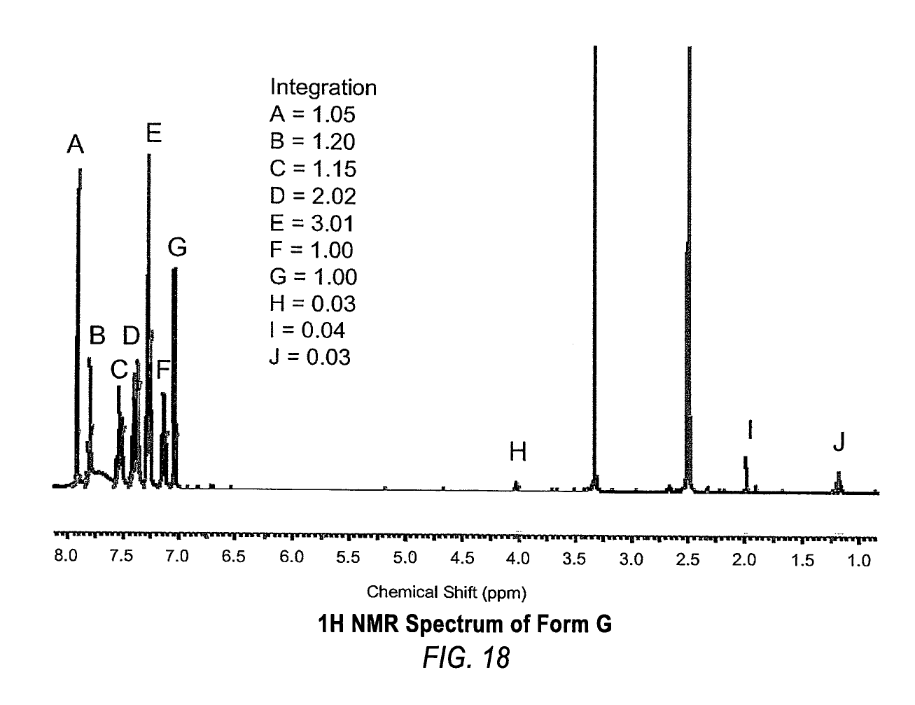

FIG. 18.

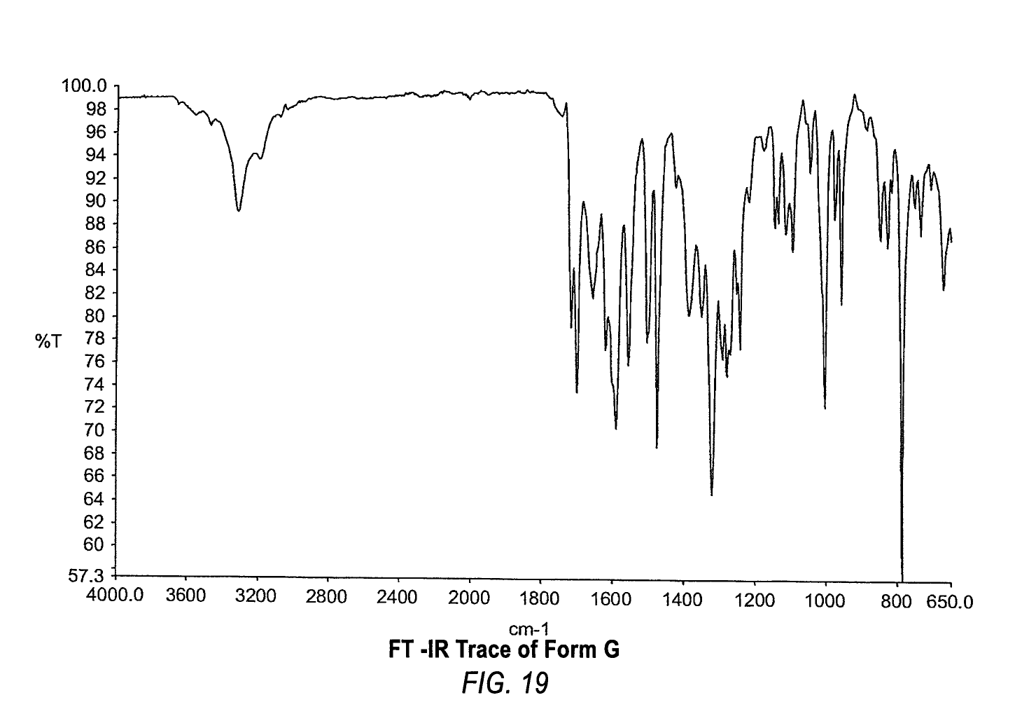

Form G can be characterized by a FT-IR spectrum as depicted in FIG. 19.

Form G can be further characterized by an endothermal event beginning at 156° C. and peaking at 163° C. as measured by DSC. Further, this coincides with a 6.5% net weight loss between 95° C. and 175° C. as measured by TGA and this can be attributed to chemical degradation. Form G can further be characterized by a second endotherm beginning at 36° C. and peaking at 61° C. as measured by DSC. This corresponds to a 2.9% net weight loss between 25° C. and 70° C. as measured by TGA.

Form G displays solubility in water of at least 0.020 mg/mL at 25° C.

Form G remains in substantially the same physical form for at least two weeks at 40° C./75% RH. Further, Form G remains chemically stable for at least two weeks at 40° C./75% RH.

Form G was found to be highly hygroscopic, displaying a total water gain of 1% (in weight) at 10% RH and a weight gain in water of more than 8% at 90% RH as seen by GVS.

Form G converted into Form F on heating above about 130° C.

Form G has been shown to convert into Form Q at a range of temperatures (e.g. from 20° C. to 50° C.), 2, 5 and 24 hours after the addition of water.

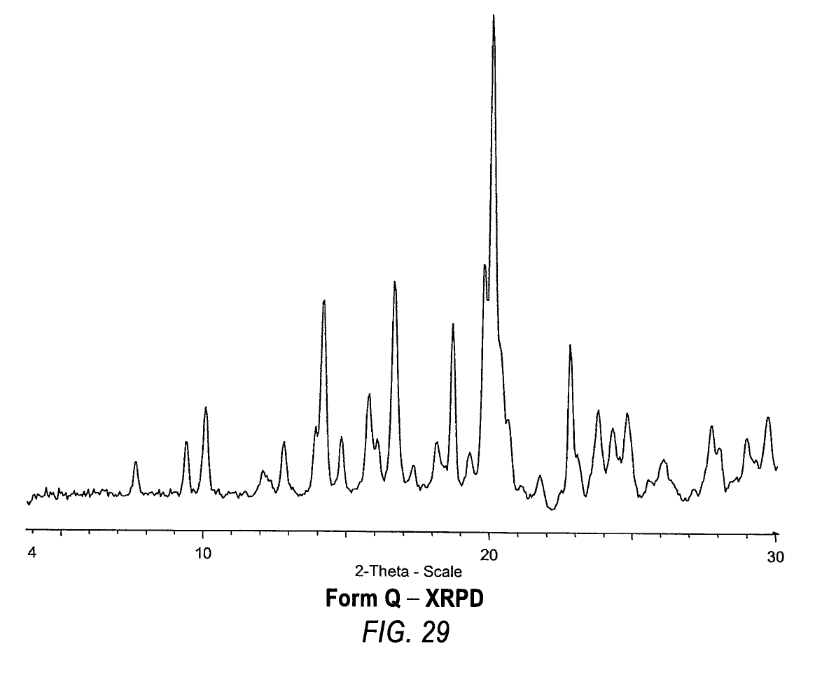

Form Q the XRPD of Form Q is shown in FIG. 29.

### Preparation and Characterization of Alternative Solid Forms Arising During Manufacture of Form C

In addition to the three neat forms of Compound I thus far described, several other forms (i.e. solvates, hydrates) have been detected and characterized during the steps leading to manufacturing of Form C.

Form A can be obtained by the multistep synthetic process depicted in Scheme II or by following the procedures described in U.S. Pat. No. 7,115,746B2, which is thereof incorporated by reference in its entirety.

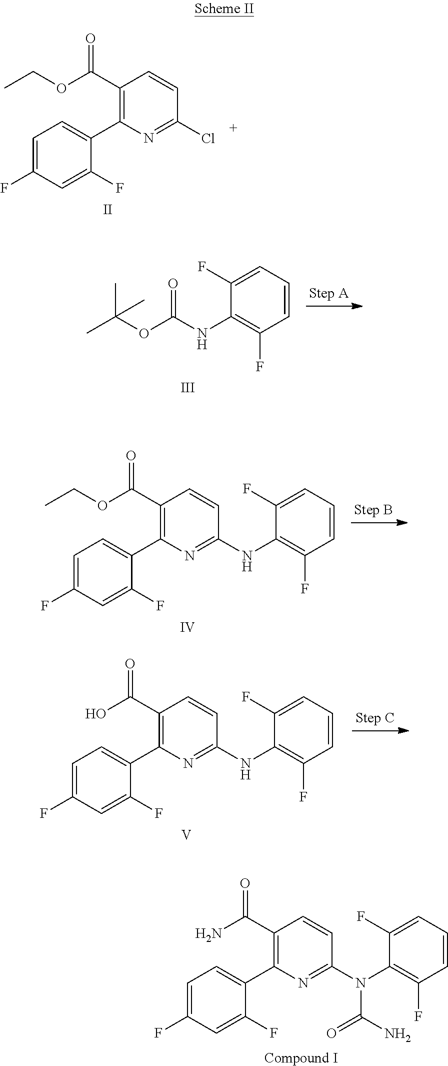

The various steps in Scheme II may be briefly described as follows:

Step A: 6-chloro-2-(2,4-difluorophenyl)-nicotinic acid ethyl ester II is available by synthesis from 2-chloronicotinic acid. Starting material II is coupled with a protected aryl amine such as Boc-2,6-difluoroaniline III in the presence of an optional transition metal catalyst such as Pd(OAc)₂, an optional ligand such as BINAP, an alkali metal salt such as cesium carbonate or K₃PO₄, in a compatible solvent such as toluene or NMP to give the Boc-protected coupling product IV. The Boc-protected coupling product IV is then reacted with an acid such as TFA in a suitable solvent such as methylene chloride to give the un-protected compound of formula IV, in the form of its HCl salt.

Step B: The ester functionality of IV is saponified in the presence of a base such as NaOH in a solvent such as THF and then acidified in the presence of an acid such as HCl to form V. Or, alternatively, the ester can be cleaved under acidic conditions, using for example HCl.

Step C: Compound V is then reacted with phosgene or diphosgene followed by NH₄OH to form the amide-urea Compound I. After work up and crystallization, the product is obtained in the crystalline solid form characterized as Form A.

Form Q is a hydrate of Form G. Both can appear as mixtures during the processes used to manufacture Form C.

Another form, Form P, was detected during the MeOH re-crystallization step used to prepare Form C from Form A. Form P is characterized below. When variable-temperature X-Ray diffraction (VT-XRD) of Form P was carried out in 5° C. increments from 25 to 50° C. and the resulting solid was cooled back to ambient conditions, it was observed that Form P transitions to Form A at approximately 40° C. and returns to Form P upon cooling back to room temperature. This suggested that Form A and Form P are enantiotropically related and that form P is the more stable of the two at room temperature. Both Form A and Form P are MeOH solvates.

### Preparation of Form A: Approaches

Form A is obtained by following the steps in Scheme II and FIG. 36, as discussed above. Forms A can be obtained as a crystalline solid (obtained from the filtrate) from Form C, by using crystallization technique M described above and MeOH as the solvent.

In a different embodiment, Form A can be obtained from Form C by crystallization methods SE or FE described herein, using MeOH as the test solvent.

### Preparation and Characterization of Form A

Representative peaks as observed in the XRPD spectrum are provided in Table V below:

TABLE V
Representative XRPD peaks for Form A

| Angle 2-θ (°) | d value (Å) | Intensity (%) |
|---|---|---|
| 13.4 | 6.59 | 58.6 |
| 14.2 | 6.21 | 76.2 |
| 15.1 | 5.87 | 71.6 |
| 17.1 | 5.18 | 67.5 |
| 19.1 | 4.65 | 100 |
| 20.1 | 4.42 | 74.4 |
| 25.0 | 3.56 | 59.1 |

Form A was shown to become crystalline Form C described herein upon slurrying with a non-solvate forming solvent such as MeOH:H₂O (1:1).

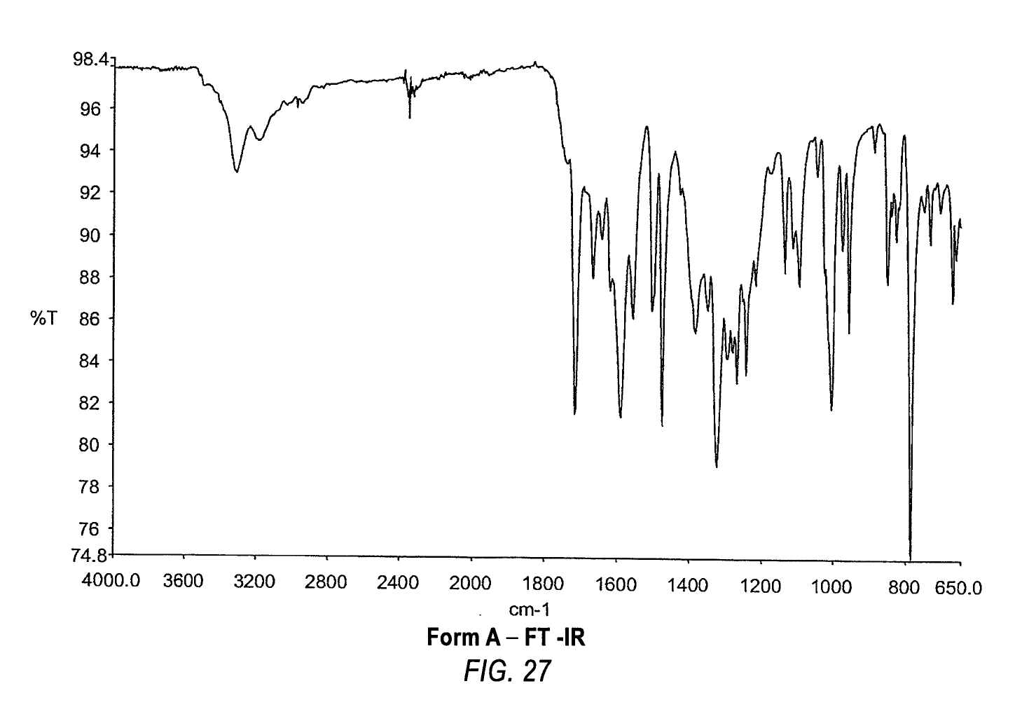

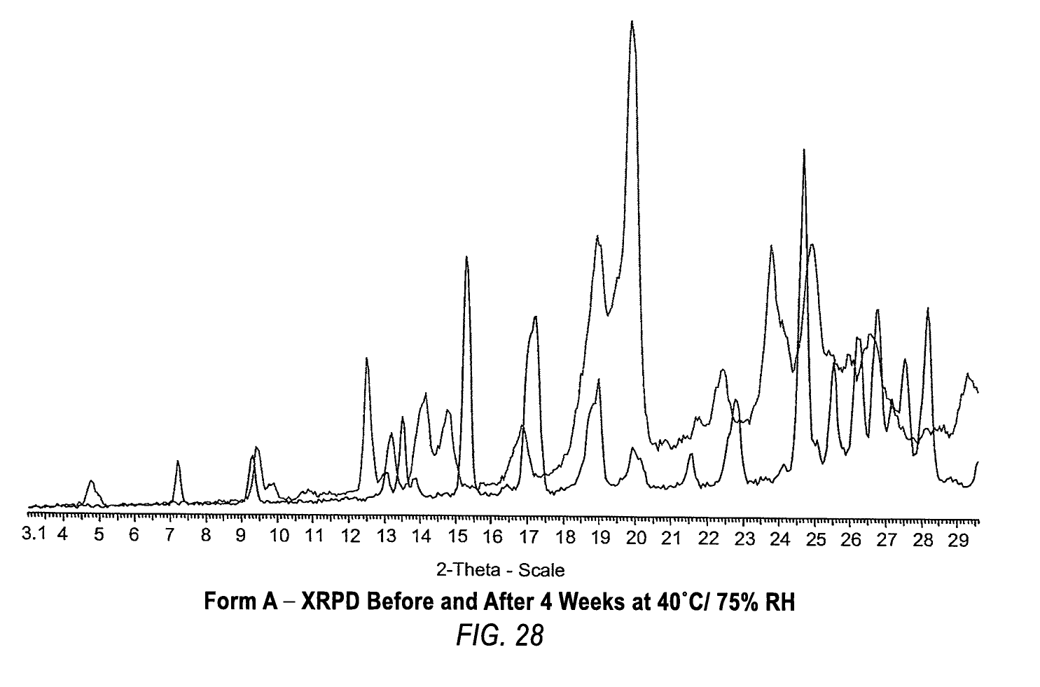

Form A can be characterized by a FT-IR spectrum as depicted in FIG. 27.

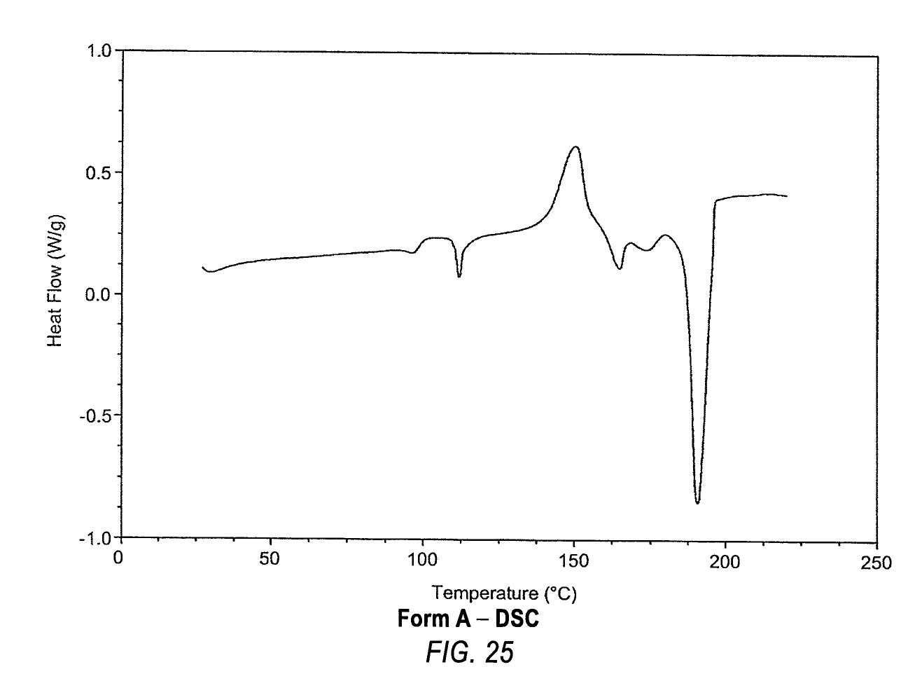

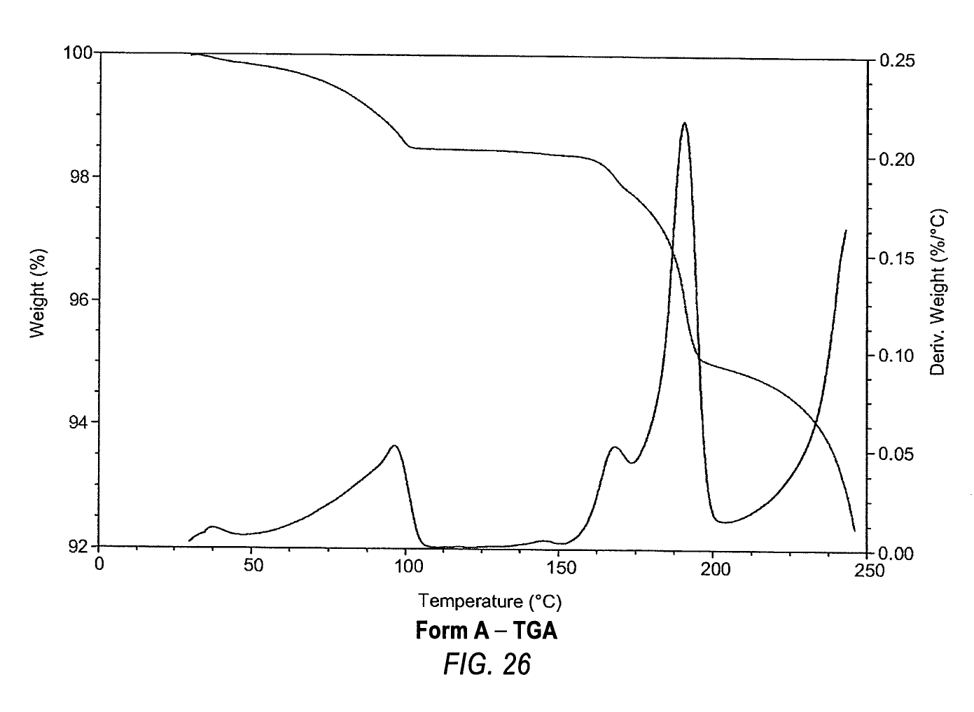

Form A can be characterized by a broad endotherm with onset 43.8° C. and peaking at 74.3° C. on DSC (FIG. 25). Further, Form A can be characterized by two other endotherms at 93° C. and 111° C., which are attributed to solvent loss (MeOH). Further, these coincide with a total weight loss of about 1.5% between 25° C. and 115° C. as seen by TGA (FIG. 26).

Form A was shown to convert to Form P upon cooling below about 50° C.

In another embodiment, Form A was shown to convert to Form G as described in FIG. 36.

### Preparation and Characterization of Form P

Form O is a crystalline form of Compound I, a mono ethyl acetate solvate, and can be obtained from Form F by slurrying Form F in ethyl acetate.

Form O, as observed by single crystal X-Ray at room temperature, has a space group P2(1)/c, with the following unit cell dimensions:
a=11.014 Å, b=26.857 Å, c=7.944 Å
α=90°, β=88.091°, γ=90°
δ_{calc} (g/cm³)=1.460.

### Preparation and Characterization of Form P

Form P is a crystalline form of Compound I and can be obtained by from Form A as shown in FIG. 36 such as by cooling below about 50° C.

In another embodiment, Form P can be obtained from Form G or Form C.

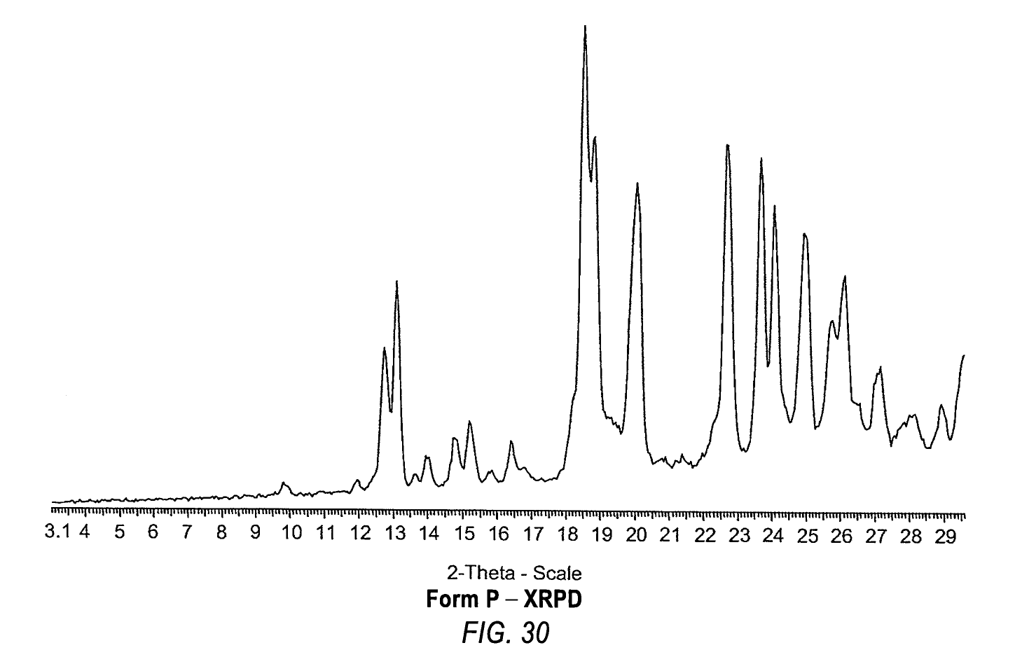

A representative XRPD pattern of Form P is provided in FIG. 30. Representative peaks as observed in the XRPD are provided in Table VI below.

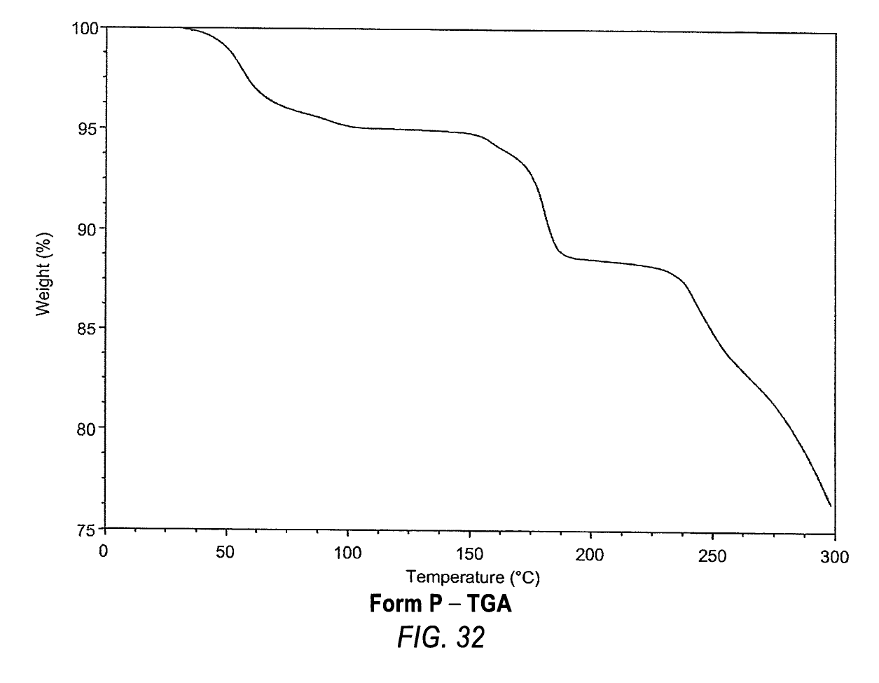

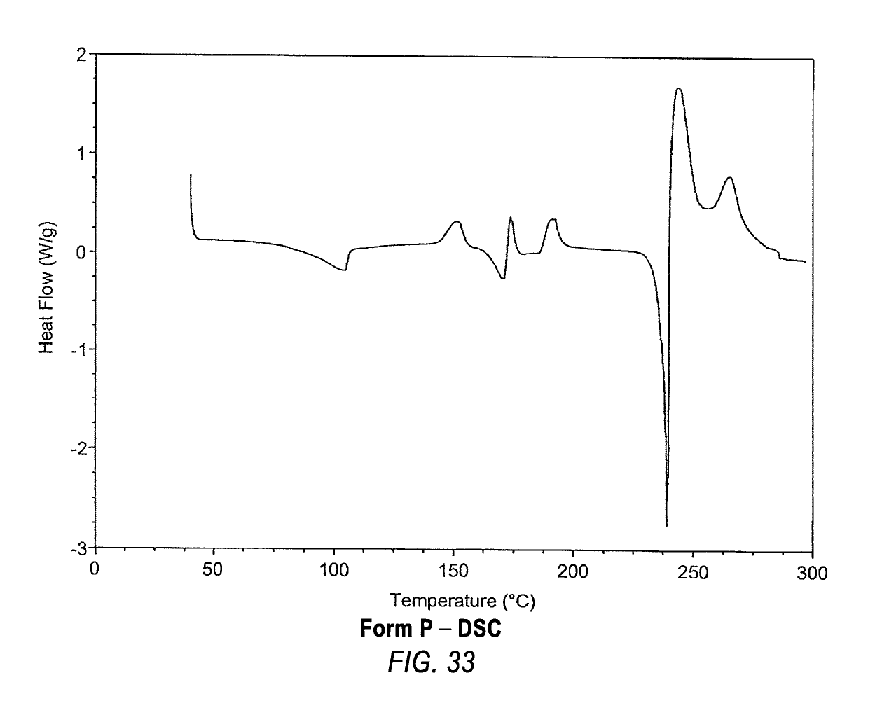

Form P can be characterized by the representative TGA and DSC traces provided in FIG. 32 and FIG. 33, respectfully.

TABLE VI
Representative XRPD peaks for Form P

| Angle 2-θ (°) | d value (Å) | Intensity (%) |
|---|---|---|
| 12.9 | 6.83 | 78.6 |
| 13.3 | 6.65 | 100 |
| 18.9 | 4.68 | 74.5 |
| 20.2 | 4.40 | 77.5 |
| 20.4 | 4.36 | 77.4 |
| 25.2 | 3.54 | 89 |
| 25.8 | 3.44 | 71.4 |

Form P has been shown to convert to Form G after 72 h of storage at 4° C. In another embodiment, Form P has been shown to convert to Form C by forming a slurry of Form P in a non-solvate forming solvent such as MeOH and water.

### Preparation and Characterization of Form Q

Form Q is a crystalline form of Compound I and it has been characterized as a 1:1 hydrate of Compound I.

Form Q can be obtained by adding water to Form G and storing the resulting solid at room temperature.

A representative XRPD pattern for Form Q is provided in FIG. 29. Representative peaks as observed in the XRPD are provided in Table VII below

TABLE VII
Representative XRPD peaks for Form Q

| Angle 2-θ | | Intensity |
|---|---|---|
| (°) | d value (\acute{Å}) | (%) |
| 9.4 | 9.36 | 50.2 |
| 10.1 | 8.75 | 54.2 |
| 12.8 | 6.89 | 50.2 |
| 14.2 | 6.21 | 66.8 |
| 14.9 | 5.96 | 50.8 |
| 15.8 | 5.59 | 55.8 |
| 16.7 | 5.30 | 68.0 |
| 18.2 | 4.88 | 50.3 |
| 18.8 | 4.73 | 64.0 |
| 19.9 | 4.47 | 71.0 |
| 20.2 | 4.40 | 100 |
| 22.9 | 3.88 | 60.0 |
| 23.8 | 3.73 | 53.7 |
| 24.3 | 3.65 | 52.0 |
| 24.9 | 3.58 | 53.5 |
| 27.8 | 3.20 | 52.4 |
| 29.0 | 3.07 | 50.9 |
| 29.8 | 3.00 | 53.4 |

### Evaluation of Solid Forms of Compound I and Methods Thereof

In one aspect, the invention provides a method of evaluating a solid form of Compound I (e.g., a solid form of Compound I, such as Forms A, C, F, G, O, P, and Q).

The method includes:

providing an evaluation of a physical or chemical parameter disclosed herein, e.g., the presence or absence of one or more peaks as measured by powder X-ray diffraction (the characteristic or value identified in this evaluation is sometimes referred to herein as a “signature”),

optionally, providing a determination of whether the value or signature (e.g., a value or signature correlated to absence or presence) for the parameter meets a preselected criteria, e.g., is present, or is present in a preselected range, and

thereby evaluating or processing the mixture.

In a preferred embodiment, the method includes providing a comparison of the value or signature with a reference, to thereby evaluate the sample. In preferred embodiments, the comparison includes determining if the test value or signature has a preselected relationship with the reference, e.g., determining if it meets the reference. The value or signature need not be numerical but can be merely an indication of whether a form is present or absent.

In a preferred embodiment, the method includes determining if a test value or signature is equal to or greater than a reference, if it is less than or equal to a reference, or if it falls with a range (either inclusive or exclusive of the endpoints of the range).

In preferred embodiments, the test value or signature, or an indication of whether the preselected relationship is met, can be memorialized, e.g., in a computer readable record.

In preferred embodiments, a decision or step is taken, e.g., the sample is classified, selected, accepted or discarded, released or withheld, processed into a drug product, shipped, moved to a new location, formulated, labeled, packaged, released into commerce, sold, or offered for sale. This can be based on whether the preselected criterion is met, e.g., based on the result of the determination of whether a signature is present, the batch from which the sample is taken can be processed.

In preferred embodiments, methods and compositions disclosed herein are useful from a process standpoint, e.g., to monitor or ensure batch-to-batch consistency or quality, or to evaluate a sample with regard to a reference, e.g., a preselected value.

In preferred embodiments, methods and compositions disclosed herein can be used to determine if a test batch of a solid form of Compound I (e.g. such as Forms A, C, F, G, O, P, and Q described herein), can be expected to have one or more of the properties of a reference or standard for the Compound I (e.g. a solid form of Compound I, such as Form A, C, F, G, O, P, and Q). Such properties can include a property listed on the product insert of an approved form of the drug, a property appearing in a compendium, e.g. the U.S. Pharmacopeia, or a property required by a regulatory agency, e.g., the U.S. Food and Drug Administration (FDA) for commercial use. A determination made by a method disclosed herein can be a direct or indirect measure of such property, e.g. a direct measure can be where the desired property is a preselected level of the subject entity being measured. In an indirect measurement, the measured subject entity is correlated with a desired characteristic, e.g., a characteristic described herein.

Some of the methods described herein include evaluating a physical or chemical parameter of a solid form of Compound I, e.g., Form A, C, F, G, O, P, and Q of Compound I. Thus, in a preferred embodiment a chemical, physical, or biological parameter disclosed herein is evaluated or determined for a solid form of Compound I, e.g., a form of a drug disclosed herein is evaluated for one or more of the following (a value or evaluation of one or more of these parameters is sometimes referred to herein as a “signature”).

The parameters include having one or more of a pre-selected:
i) A powder X-ray diffraction pattern peak or peaks;
ii) an endotherm or Tₘ, e.g., as measured in DSC;
iii) a value of weight gain or loss at a certain temperature or temperature range as determined by TGA.
iv) a value for weight gain, e.g., from 5 to 95% relative humidity at 25° C. as measured using GVS;
v) a value for the solubility in water;
vi) measure of the ability to remain in substantially the same physical or chemical form under preselected conditions;
vii) a ¹H NMR pattern peak or peaks;
viii) a FT-IR spectrum trace as disclosed herein;
ix) a specific single crystalline space group; and unit cell dimensions disclosed herein as determined by single crystal X-Ray crystallography.

### Formulation, Uses and Administration

### Pharmaceutically Acceptable Compositions

Pharmaceutically acceptable compositions of this invention comprise solid forms of Compound I described herein (e.g. crystalline neat solid forms, salts or solvates) and a pharmaceutically acceptable carrier, adjuvant, or vehicle. The amount of the solid form or solid forms of Compound I in the compositions of this invention is such that it is effective to measurably inhibit a protein kinase, particularly p38, in a biological sample or in a patient. Preferably the composition of this invention is formulated for administration to a patient in need of such composition. Most preferably, the composition of this invention is formulated for oral administration to said patient.

The term “measurably inhibit”, as used herein means a measurable change in kinase activity, particularly p38 kinase activity, between a sample comprising a compound of this invention and p38 kinase and an equivalent sample comprising p38 kinase in the absence of said compound.

The term “patient”, as used herein, means an animal, preferably a mammal, and most preferably a human.

The term “pharmaceutically acceptable carrier” refers to a non-toxic carrier that may be administered to a patient, together with a solid form of Compound I described herein (e.g. a neat solid form, a salt or a solvate), and which does not destroy the pharmacological activity thereof.

Accordingly, in another aspect of the present invention, pharmaceutically acceptable compositions are provided, wherein these compositions comprise any of the solid forms of Compound I as described herein, and optionally comprise a pharmaceutically acceptable carrier, adjuvant or vehicle. Further, in certain embodiments, these compositions optionally comprise one or more additional therapeutic agents. Such agents include but are not limited to an antibiotic, an anti-inflammatory agent, an analgesic, a matrix metalloprotease inhibitor, a lipoxygenase inhibitor, a cytokine antagonist, an immunosuppressant, an anti-cancer agent, an anti-viral agent, a cytokine, a growth factor, an immunomodulator, a prostaglandin, an anti-rheumatic medication or an anti-vascular hyperproliferation compound.

In another embodiment, the additional therapeutic agent can be selected from an anti-inflammatory agent, an analgesic, an anti-cancer agent, an anti-proliferative compound, an anti-rheumatic agent, an agent used to inhibit thrombin-induced platelet aggregation, an immunomodulator, an agent to treat the symptoms of allergies or an agent to treat destructive bone diseases (e.g. post-menopausal osteoporosis).

In yet another embodiment, the composition including a solid form of Compound I, can be administered in combination with an additional anti-inflammatory agent, an analgesic or an anti-rheumatic agent. Anti-inflammatory agents can be selected from, but not limited to: a steroidal anti-inflammatory drug such as a glucocorticoid (e.g. hydrocortisone, prednisone, prednisolone, methylprednisolone, cortisone acetate, betamethasone, triamcinolone, beclometasone, fludrocortisone acetate (Florinef®), deoxycorticosterone acetate, aldosterone, dexamethasone), a non-steroidal anti-inflammatory drug (e.g. aspirin and other salicylates, ibuprofen and other profens (e.g. naproxen), diclofenac and other arylalkanoic acids, fenamic acids (e.g. Meclofenamic acid), pyrazolidine derivatives (e.g. Metamizole), oxicams (e.g. Piroxicam), nimesulide, licofelone.

Said analgesic can be selected from, but not limited to: acetamidophen (or paracetamol in Europe), a COX-2 inhibitor (e.g. celecoxib), an opiate or morphinomimetic (e.g., codeine, oxycodone, hydrocodone, diaorphine, pethidine, buprenorphine). diproqualone, lidocaine,

Said anti-rheumatic agents can be selected from, but not limited to: azathioprine, cyclosporine A, D-penicillamine, gold salts, hydroxychloroquine, leflunomide, methotrexate, minocycline, sulfasalazine, TNF-α blockers (e.g. Enbrel®, Remicade®, Humira®), Interleukin-1 blockers, monoclonal antibiotics against B cells (e.g. Rituxan®), T-cell activation blockers (e.g. Orencia®)

It will also be appreciated that certain of the compounds of the present invention can exist in free form for treatment.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the solid form of Compound I of the invention, such as by producing any undesirable biological effect or otherwise interacting in a deleterious manner with any other component(s) of the pharmaceutically acceptable composition, its use is contemplated to be within the scope of this invention. In some cases, the pH of the formulation may be adjusted with pharmaceutically acceptable acids, bases or buffers to enhance the stability of the formulated compound or its delivery form.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Most preferably the compositions are administered orally. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one aspect, the invention features a composition (or pharmaceutical composition) wherein essentially all of the Compound I is in a first solid form disclosed herein, determined by for example evaluating physical or chemical parameter disclosed herein.

In another aspect, the invention features a composition (or pharmaceutical composition) comprising a first solid form of the Compound I described herein as determined, e.g., by evaluating physical or chemical parameter disclosed herein and a second solid form of Compound I, determined, e.g., by evaluating a physical or a chemical parameter disclosed herein. In some embodiments, the first and second solid forms comprise at least one homogenous portion, i.e., regions enriched for one of the said solid forms. In other embodiments, the first and second solid forms of Compound I are heterogenous within the composition.

In one aspect, the invention features a pharmaceutical composition comprising a solid form of 2-(2,4-difluorophenyl)-6-(1-(2,6-difluorophenyl)ureido)nicotinamide described herein and a pharmaceutically acceptable excipient. In some embodiments, the composition is an aqueous solution. In some embodiments, the composition comprises a solid. In some embodiments, the composition is an oral suspension. In some embodiments, the composition is a solid oral dosage form (e.g., a tablet or capsule).

### Uses of the Compounds and Compositions of the Invention

The solid forms of Compound I described herein are useful generally for inhibiting p38 kinase in biological samples or in a patient. In another embodiment, the invention comprises a method of treating or lessening the severity of a p38-mediated condition or disease in a patient. The term “p38 mediated disease”, as used herein means any disease or other deleterious condition in which in particular p38 is known to play a role. The term “biological sample”, as used herein, means an ex vivo sample, and includes, without limitation, cell cultures or extracts thereof; tissue or organ samples or extracts thereof, biopsied material obtained from a mammal or extracts thereof; and blood, saliva, urine, feces, semen, tears, or other body fluids or extracts thereof.

In another embodiment, the solid forms of Compound I and their pharmaceutically acceptable compositions described herein are useful for the treatment of acute and chronic inflammatory diseases, cancer, autoimmune disease, immunodeficiency disorders, destructive bone disorders (e.g., post-menopausal osteoporosis), proliferative disorders, infectious diseases, viral diseases, allergies, asthma, burns and neurodegenerative diseases. These solid forms and compositions are also useful in methods for preventing cell death and hyperplasia and therefore might be used to treat or prevent reperfusion/ischemia in stroke, heart attacks, organ hypoxia. These solid forms and compositions are also useful in methods for preventing thrombin-induced platelet aggregation.

The term “treatment”, as used herein, unless otherwise indicated, means the treatment of a disorder or disease as provided in the methods described herein, including curing, reducing the symptoms of or slowing the progress of said disorder. The terms “treat” and “treating” are defined in accord with the foregoing term “treatment”.

Inflammatory diseases that can be treated include but are not limited to rheumatoid arthritis (RA), psoriasis, Crohn's Disease, psoriatic arthritis, ulcerative colitis and ankyosing spondylitis, other forms of inflammatory bowel disease, acute idiopathic polyneuritis, lupus, optic neuritis, temporal artheritis, acute and chronic pancreatitis, neuritischronic pulmonary obstruction and burns.

Autoimmune diseases which may be treated include, but are not limited to glomeralonephritis, scleroderma, chronic thyroiditis, Graves' disease and graft vs. host disease.

Destructive bone disorders which may be treated include, but are not limited to osteoporosis, osteoarthritis, and multiple myelonoma-related bone disorder.

Proliferative disorders which may be treated include but are not limited to, acute myelogeneous leukemia, chronic myelogeneous leukemia, metastatic melanoma, Kaposi's sarcoma, and multiple myeloma.

Infectious diseases that may be treated include, but are not limited to sepsis, septic shock and Shigellosis.

Viral diseases that may be treated include, but are not limited to, acute hepatitis infection (including hepatitis A, B and C), HIV infection and CMV retinitis.

Degenerative diseases which might be treated include, but are not limited to Alzheimer's disease, Parkinson's disease and cerebral ischemia.

In another aspect, methods and compositions disclosed herein can be used where the presence, distribution, or amount, of one or more solid forms of Compound I in the mixture may possess or impinge on the biological activity. The methods are also useful from a structure-activity prospective, to evaluate or ensure biological equivalence.

The compositions of this invention, comprising one or more solid forms of Compound I may be employed in a conventional manner for treating chronic inflammatory diseases, cancer, autoimmune disease, immunodeficiency disorders, destructive bone disorders (e.g., post-menopausal osteoporosis), proliferative disorders, infectious diseases, viral diseases, allergies, asthma, burns and neurodegenerative diseases in vivo and in a patient. Such methods of treatment, their dosage levels and requirements may be selected by those ordinarily skilled in the art from available methods and techniques.

### Administration of Compounds and Compositions of the Invention

In some embodiments, a solid form of Compound I described herein is administered as a composition, for example a solid, liquid (e.g., a suspension), or an iv (e.g., a solid form of compound I is dissolved into a liquid and administered iv).

In some embodiments, the composition is administered with an additional therapeutic agent, such as those described above in order to increase the effect of the therapy against said disease. The additional therapeutic agent, for example one described above, can be administered as a composition, for example a solid, liquid (e.g., a suspension), or an iv (e.g., a form of compound one is dissolved into a liquid and administered iv). The additional agent can be administered before (e.g., about 1 day, about 12 hours, about 8 hours, about 6 hours, about 4 hours, about 2 hours, about 1 hour, about 30, or about 15 minutes or less), during, or after (e.g., about 15 minutes, about 30 minutes, about 1 hour, about 2 hours, about 4 hours, about 6 hours, about 8 hours, about 12, hours, or about 1 day, or more) the administration of the composition comprising a solid form of Compound I. In some embodiments, the composition including a solid form of Compound I also includes the additional therapeutic agent, for example, a solid, liquid (e.g., a suspension), or an iv (e.g., a form of compound one is dissolved into a liquid and administered iv) composition includes a solid form of Compound I described herein and at least one additional therapeutic agent such as an anti-inflammatory agent, for example one described above.

The pharmaceutical compositions of this invention may be administered orally, parenterally, by inhalation spray, topically, via ophthalmic solution or ointment, rectally, nasally, buccally, vaginally or via an implanted reservoir. The pharmaceutical compositions of this invention may contain any conventional non-toxic pharmaceutically-acceptable carriers, adjuvants or vehicles. In some cases, the pH of the formulation may also be adjusted with pharmaceutically acceptable acids, bases or buffers to enhance the stability of the formulated compound or its delivery form. The term “parenteral” as used herein includes subcutaneous, intracutaneous, intravenous, intramuscular, intra-articular, intrasynovial, intrasternal, intrathecal, intralesional and intracranial injection or infusion techniques.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and solutions and propylene glycol are administered orally, the active ingredient is combined with emulsifying and suspending agents. If desired, certain sweetening and/or flavoring and/or coloring agents may be added.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions of this invention may also be administered in the form of suppositories for rectal or vaginal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-administered transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the Form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment.

Dosage levels of between about 0.01 and about 100 mg/kg body weight per day, preferably between 0.5 and about 75 mg/kg body weight per day and most preferably between about 1 and 50 mg/kg body weight per day of the active ingredient solid form of Compound I are useful in a monotherapy for the treatment of an inflammatory disease such as RA, psoriasis, Crohn's Disease, psoriatic arthritis, ulcerative colitis and ankyosing spondylitis, other forms of inflammatory bowel disease, acute idiopathic polyneuritis, lupus, optic neuritis, temporal artheritis, acute and chronic pancreatitis, neuritischronic pulmonary obstruction and burns.

Typically, the pharmaceutical compositions of this invention will be administered from about 1 to 5 times per day or alternatively, as a continuous infusion. Such administration can be used as a chronic or acute therapy. The amount of active ingredient that may be combined with the carrier materials to produce a single dosage form will vary depending upon the disease treated and the particular mode of administration. A typical preparation will contain from about 5% to about 95% active compound, e.g. a solid form of Compound I described herein (w/w). Preferably, such preparations contain from about 20% to about 80% active compound.

When the compositions of this invention comprise a combination of a solid form of Compound I, and one or more additional therapeutic agents, both the solid form of Compound I and the additional therapeutic agent should be present at dosage levels of between about 10% to 80% of the dosage normally administered in a monotherapy regime.

Upon improvement of a patient's condition, a maintenance dose of a compound, composition or combination of this invention may be administered, if necessary. Subsequently, the dosage, dosage form, or frequency of administration, or both, may need to be modified. In some cases, patients may, however, require intermittent treatment on a long-term basis upon any recurrence or disease symptoms.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, and the patient's disposition to the disease and the judgment of the treating physician.

One embodiment of this invention provides a method for treating a disease (e.g. an inflammatory disease such as such as RA, psoriasis, Crohn's Disease, psoriatic arthritis, ulcerative colitis and ankyosing spondylitis, other forms of inflammatory bowel disease, acute idiopathic polyneuritis, lupus, optic neuritis, temporal artheritis, acute and chronic pancreatitis, neuritischronic pulmonary obstruction and burns) in a subject comprising the step of administering to the subject any compound, pharmaceutical composition, or combination described herein and a pharmaceutically acceptable carrier, e.g. a pharmaceutically acceptable carrier described above.

According to another embodiment, a form of Compound I described herein may also be delivered by implantation (e.g., surgically), such as with an implantable or indwelling device. An implantable or indwelling device may be designed to reside either permanently or temporarily in a subject. Examples of implantable and indwelling devices include, but are not limited to, contact lenses, central venous catheters and needleless connectors, endotracheal tubes, intrauterine devices, mechanical heart valves, pacemakers, peritoneal dialysis catheters, prosthetic joints, such as hip and knee replacements, tympanostomy tubes, urinary catheters, voice prostheses, stents, delivery pumps, vascular filters and implantable control release compositions. In addition, implantable or indwelling devices may be used as a depot or reservoir of Compound I. Any implantable or indwelling device can be used to deliver Compound I provided that a) the device, Compound I and any pharmaceutical composition including Compound I are biocompatible, and b) that the device can deliver or release an effective amount of Compound Ito confer a therapeutic effect on the treated patient.

Delivery of therapeutic agents via implantable or indwelling devices is known in the art. See for example, “Recent Developments in Coated Stents” by Hofma et al. published in Current Interventional Cardiology Reports 2001, 3:28-36, the entire contents of which, including references cited therein, are incorporated herein. Other descriptions of implantable devices can be found in U.S. Pat. Nos. 6,569,195 and 6,322,847; and U.S. Patent Application Numbers 2004/0044405, 2004/0018228, 2003/0229390, 2003/0225450, 2003/0216699 and 2003/0204168, each of which is incorporated herein in its entirety.

In some embodiments, the implantable device is a stent. In one specific embodiment, a stent can include interlocked meshed cables. Each cable can include metal wires for structural support and polyermic wires for delivering the therapeutic agent. The polymeric wire can be dosed by immersing the polymer in a solution of the therapeutic agent. Alternatively, the therapeutic agent can be embedded in the polymeric wire during the formation of the wire from polymeric precursor solutions.

In other embodiments, implantable or indwelling devices can be coated with polymeric coatings that include the therapeutic agent. The polymeric coating can be designed to control the release rate of the therapeutic agent. Controlled release of therapeutic agents can utilize various technologies. Devices are known that have a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable or indwelling device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The device can be coated by spraying the device with a solution or dispersion of polymer, active agent, and solvent. The solvent can be evaporated, leaving a coating of polymer and active agent. The active agent can be dissolved and/or dispersed in the polymer. In some embodiments, the co-polymers can be extruded over the device.

### EXAMPLES

As used herein, all abbreviations and conventions used throughout this application are consistent with those in contemporary scientific literature. See e.g. Janet S. Dodd, ed., The ACS Style Guide: A Manual for Authors and Editors, 2^{nd} Ed., Washington, D.C.: American Chemical Society, 1997, herein incorporated by reference in its entirety.

A number of embodiments of the invention are exemplified in the following section. Nevertheless, it will be understood that various modifications may be made without departing from the spirit and scope of the invention. Accordingly, other embodiments are within the scope of the invention.

### Description of Physical Characterization Techniques Used

### X-Ray Powder Diffraction (XRPD)

All patterns were collected in one of the five systems described below:

1. Bruker D8 Discover with HighStar array detector with an accelerator voltage of 40 kV and current of 35 mA over a 120 second acquisition time. Each sample was prepared in a nickel sample holder and the subsequent pattern collected over two frames with a 2θ range of 4-41°. Variable temperature X-Ray diffraction (VT-XRD) was accomplished with a DHS900 Anton Paar heating stage with a TCU150 controller with a heating rate between steps of 10° C./min and an equilibration time of 5 minutes at temperature.
2. Siemens D5000 diffractometer using CuKα radiation (40 kV, 40 mA), θ-θ goniometer, automatic divergence and receiving slits, a graphite secondary monochromator and a scintillation counter. The data were collected over an angular 2 theta range of 2° to 42° in continuous scan mode using a step size of 0.02° and a step time of 1 second. Samples were run under ambient conditions and prepared as flat plate specimens using powder as received without grinding. Approximately 1-2 mg of the sample was slightly pressed on a glass slide to obtain a flat surface. Samples run under non-ambient conditions were mounted on a silicon wafer with heat conducting compounds. The sample was then heated to the appropriate temperature at ca. 20° C./min and subsequently held isothermally for ca 1 minute before data collection was initiated.
3. Bruker AXS C2 GADDS Diffractometer using CuKα radiation (40 kV, 40 mA), automated XYZ stage, laser video microscope for auto-sample positioning and a HiStar 2-dimensional area detector. X-Ray optics consists of a single Gobel multilayer mirror coupled with a pinhole collimator of 0.3 mm. Beam divergence, was approximately 4 mm. A θ-θ continuos scan mode was employed with a sample to detector distance of 20 cm which gives an effective 2θ range of 3.2-29.8°. A typical exposure time of a sample in this system would be 120 s. Samples were run under ambient conditions and prepared as flat plate specimens using powder as received without grinding. Approximately 25-50 mg of the sample was gently packed into 12 mm diameter, 0.5 mm deep cavities cut into polished, zero-background (510) silicon wafers (from The Gem Dugout). All specimens were run both stationary and rotated on their own plate during analysis. A further specimen was run using silicon powder as internal standard to correct for any peak displacement. Diffraction data were reported using Cu Kαₗ (l=1.5406 Å), after the Kαₗ component had been stripped using EVA, the powder patterns were indexed by the ITO method using WIN-INDEX and the raw lattice constants refined using WIN-METRIC.
4. Shimadzu XRD-6000 X-Ray powder diffractometer using CuKα radiation (40 kV, 40 mA) equipped with a fine-focus X-Ray tube. Divergence and Scattering slids were set at 1° and the receiving slit was set at 0.15 mm. A NaI scintillation detector detected diffracted irradiation. A θ-2θ continuous scan at 3°/min (0.4 sec/0.02° step) from 2.5° to 40° was used. A silicon standard was analyzed each day to check the instrument alignment. Each sample was prepared for analysis by pressing it onto the sample holder.
5. Inel XRG-3000 X-Ray powder diffractometer using CuKα radiation (40 kV, 30 mA). This was equipped with a curved position-sensitive detector. Data was collected in real time over a 2 θ range of 120° at a resolution of 0.03°. Samples were packed in an aluminum holder with a silicon insert and analyzed. A silicon standard was analyzed each day to check for instrument alignment. Only the 2 theta region between 4° and 40° is shown for data run on this instrument.

### Differential Scanning Calorimetry (DSC)

DSC were collected using one of the two instruments described below:

1. TA Instrument Q1000 series mDSC with standard aluminium hermetic pans and lids that were punctured with a pin-hole. Each sample was ramped at 10° C./min from 35° C. to 200° C. or from 10° C. to 300° C. with no modulation. The energy and temperature calibration standard was indium. A nitrogen purge at 30 mL/min was maintained over the sample. Between 1 and 3 mg of sample was used.
2. TA 2920 instrument, using indium as a calibration standard, with crimped pans with one pinhole. Approximately 2-5 mg samples were placed into a DSC pan, and the weight accurately recorded. Samples were heated under nitrogen at a rate of 10° C./min, up to a final temperature of 350° C.

### Thermogravimetric Analysis (TGA)

TGA were collected using one of two instruments described below:

1. TA Instruments Q5000 series TGA with crimped aluminum sample pans. Each sample was ramped at 10° C./min from ambient to 300° C. The system was calibrated with Nickel/Alumel. A nitrogen purge of 60 mL/min was maintained over the sample. Typically 2-10 mg of sample was loaded onto a pre-tared platinum crucible.
2. TA instruments TGA 2050. Nickel and alumel calibration standards were used. Approximately 5.0 mg of sample was placed in the pan, accurately weighed and inserted into the TG furnace. Samples were heated in nitrogen at a rate of 10° C./min, up to a final temperature of 300-350° C.

### Gravimetric Vapour Desorption (GVS) Studies

All samples were run on a Hiden IGASorp moisture sorption analyzer running CFRSorp software. Sample sizes were typically 10 mg. A moisture absorption-desorption isotherm was performed as outlined below in Table X (2 scans giving 1 complete cycle). All samples were loaded/unloaded at typical room humidity and temperature (40% RH, 25° C.). All samples were analysed by XRPD post GVS analysis. The standard isotherm was performed at 25° C. at 10% RH intervals over a 0-90% RH range.

TABLE X

| | |
|---|---|
| Scan1 | Scan2 |

| Adsorption | Desorption | Adsorption |
|---|---|---|
| 40 | 85 | 10 |
| 50 | 75 | 20 |
| 60 | 65 | 30 |
| 70 | 45 | 40 |
| 80 | 35 | |
| 90 | 25 | |
| | 15 | |
| | 5 | |
| | 0 | |

### Infra-Red Spectroscopy; ATR-IR and TG-IR

One of the three systems described below was used:

1. Perkin-Elmer Spectrum fitted with a Universal ATR sampling accessory. Data collected and analyzed using Spectrum V5.0.1 software.
2. Seiko Instruments TG/DTA 220 interfaced with a Nicolet model 560 Fourier transForm IR spectrophotometer, equipped with a globar source, Ge/KBr beamsplitter, and deuterated triglycine sulfate (DTGS) detector. The IR spectrophotometer was wavelength calibrated weekly, using nickel and alumel for the temperature calibration. Samples of approximately 10 mg were weighed into a platinum pan and heated from 30° C. to 300° C. at a rate of 20° C. with a helium purge. IR spectra were obtained in series, with each spectrum representing 32 co-added scans at a resolution of 4 cm⁻¹. Spectra were collected with a 33-second repeat time. Volatiles were identified from a search of the HR Nicolet TGA vapor phase spectral library.
3. mid-IR spectra were acquired on a Nicoled model 860 Fourier transForm IR spectrophotometer equipped with a globar source, Ge/KBr beamsplitter, and deuterated triglycine sulfate (DTGS). A spectra-Tech, Inc. diffuse reflectance accessory was utilized for sampling. Each spectrum represents 128 co-added scans at a spectral resolution of 4 cm⁻¹. A background data set was then acquired. Subsequently, a Log 1/R (R=reflectance) spectrum was acquired by rationing the two data against each other. The spectrophotometer was calibrated for wavelength with polystyrene at the time of use.

### Solubility Analysis (in Water)

This was measured by suspending enough Compound I in 0.25 mL of solvent (water) to give a maximum final concentration of ≧10 mg/ml of the parent free form of the compound. The suspension was equilibrated at 25° C. for 24 hrs followed by a pH check and filtration through a glass fibre C96 well plate. The filtrate was then diluted down by a factor of 101. Quantitation was by HPLC (Table XI) with reference to a standard, diluted and undiluted tests were injected. The solubility was calculated by integration of the peak area found at the same retention time as the peak maximum in the standard injection. If there is sufficient solid in the filter plate the XRPD is normally checked for phase changes, hydrate formation, amorphization, crystallization, etc.

TABLE XI
HPLC gradient conditions.

| Time/min | % Phase A | % Phase B |
|---|---|---|
| 0.0 | 95 | 5 |
| 1.0 | 80 | 20 |
| 2.3 | 5 | 95 |
| 3.3 | 5 | 95 |
| 3.5 | 95 | 5 |
| 4.4 | 95 | 5 |

### ¹H NMR

All spectra were collected on a Bruker 400 MHz system equipped with an autosampler. Samples were prepared in d₆-DMSO, unless otherwise stated.

### Karl-Fisher Water Determination Studies

Water content was measured on a Mettler Toledo DL39 Coulometer using Hydranal Coulomat AG reagent and an Argon purge.

### Purity Analysis (by HPLC)

Purity analysis was performed on an Agilent HP1100 series system equipped with a diode array detector and using ChemStation software v9. The specific conditions are collected in Table XII.

TABLE XII
HPLC conditions used for purity determination.

| | | | |
|---|---|---|---|
| Type of method | Normal Phase | Reverse Phase | ✓ |
| | Isocratic | Gradient | ✓ |

| Column: | Agilent Zorbax SB-Phenyl 150 × 4.6 mm, 5 μm | | |
|---|---|---|---|
| Column | 40 | | |
| Temperature/° C.: | | | |
| Injection/μl: | 10 | | |
| Detection: | 215, 8 | | |
| Wavelength, | | | |
| Bandwidth/nm: | | | |
| Flow Rate/ml/min: | 1.5 | | |
| Phase A: | Water:acetonitrile:trifluoroacetic acid (85:15:0.05) | | |
| Phase B: | Water:acetonitrile:trifluoroacetic acid (30:70:0.05) | | |
| | Time/Min | % Phase A | % Phase B |
| Timetable: | 0 | 100 | 0 |
| | 2 | 100 | 0 |
| | 6 | 78 | 22 |
| | 35 | 27 | 73 |
| | 40 | 27 | 73 |
| | 41 | 100 | 0 |
| | 49 | 100 | 0 |

Although specific details such as instrument model, equipment settings and conditions, are described herein, one skilled in the art will appreciate that each analytical experiment includes instrument and human errors. Additionally, the foregoing is not meant to limit the performance of experiments to specific instruments and/or equipment settings. Moreover, the exact outcome or measured values of each experiment depend upon representative sampling and how the sample is maintained before and during physical characterization. Differences in representative sampling and/or sample maintenance may result in variations in exact outcome or measured values of each experiment.

As described herein, all 2 theta values should be interpreted to be the reported value +/−0.2 degrees. For example, a XPRD spectra with an annotated peak position of 9.5 degrees 2 theta represents a peak position of 9.3 to 9.7 degrees 2 theta (i.e., 9.5 +/−0.2 degrees 2 theta).

### General Synthetic Schemes

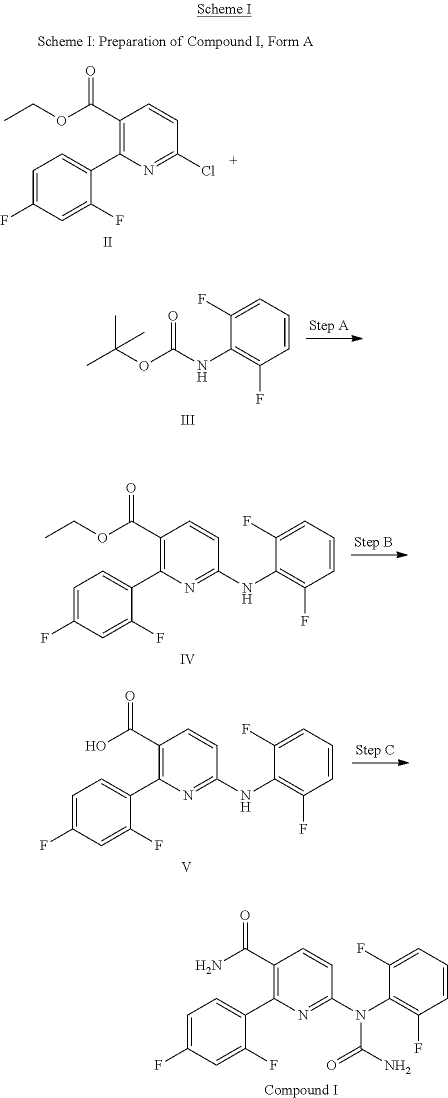

### Preparation of Starting Materials II and III

### Preparation of 2-(2,4-difluorophenyl)-nicotinic acid ethyl ester (VI)

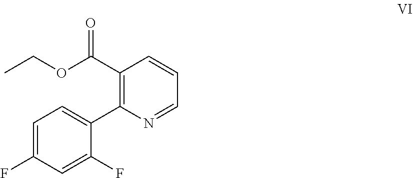

To a nitrogen purged 3.0 L, 4-necked flask, fitted with an overhead stirrer, thermocouple, heating mantle, nitrogen outlet and reflux condenser, was charged Pd(Ph₃)₄ (5.0 g, 4.33 mmoles, 0.005 eq), sodium carbonate (92.6 g, 874 mmoles, 1.3 eq), ethyl 2-chloronicotinate (126.0 g, 678 moles, 1.0 eq), 2,4-difluorophenylboronic acid (125 g, 791 mmoles, 1.2 eq), followed by 0.5 L of toluene and 125 mL denatured EtOH. The reaction was heated to 82° C. with vigorous stirring under N₂ overnight. HPLC analysis [Tᵣₑₜ SM=10 min, Tᵣₑₜ VI=12 min] of the reaction mixture showed that the starting material was completely consumed and a later-eluting peak produced. (by TLC R_{f}=0.4 using 2:1 hexanes:ethyl acetate). The reaction was cooled to room temperature, the mixture filtered through a small pad of Celite® and the solvents removed under vacuum at 55° C. The residue was dissolved in EtOAc, washed, dried (MgSO₄), filtered through Celite® again, and concentrated. The product was obtained as a yellow solid (162 g, 91.0% yield).

### Preparation of 2-(2,4-difluorophenyl)-1-oxy-nicotinic acid ethyl ester (VII)

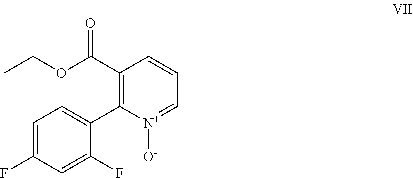

To a nitrogen purged, 12 L, 5-necked flask, fitted with an overhead stirrer, a thermocouple and a condenser, was charged the diaryl pyridine VI (144 g, 548 mmoles, 1.0 eq) and 4 L of CH₂Cl₂. With stirring, the m-CPBA was added over 5 minutes. The temperature gradually increased from 22 to 38° C. in 45 minutes. Vigorous stirring was continued under nitrogen until the HPLC analysis [Tᵣₑₜ VI=12 min, Tᵣₑₜ VII=10 min] showed >97% completion. The reaction was cooled to room temperature and the contents slowly poured onto 3 L of water. Added Na₂SO₃ slowly (exotherm from 20 to 33° C.), until the peroxide test (starch/I₂ paper) indicated no peroxides remaining in the mixture. Removed the aqueous layer and washed the organic layer with satd. NaHCO₃ (about 3 L). The combined organics were dried (MgSO₄), filtered, and concentrated to a brown thick oil. This was stirred in MTBE (2 L) to give a white precipitate. This was collected by filtration, washed with MTBE and dried under vacuum to give intermediate compound VII. (692 g, 67% yield).

### Preparation of 6-Chloro-2-(2,4-difluorophenyl)-nicotinic acid ethyl ester (II)

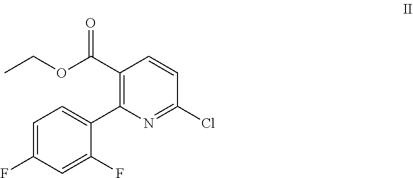

To a nitrogen purged 500 mL, 3-necked flask, fitted with a reflux condenser, heating mantle and a thermocouple was charged the N-Oxide VII (21 g, 75 mmoles, 1.0 eq) followed by 150 mL dichloroethane. The phosphorous oxychloride (75 mL) was added all at once with stirring, causing an immediate rise in temperature from 21 to 23° C. followed by gradual warming after that. The solution was heated under nitrogen to 70-75° C. until HPLC analysis [Tᵣₑₜ VII=10 min, Tᵣₑₜ II=17 min] showed >94% completion. The reaction was cooled to room temperature and the contents concentrated under vacuum to remove most of the POCl₃. The remainder was quenched by slowly pouring onto 450 g of ice. After melting the ice, the product was extracted into methylene chloride (2×200 mL). The combined organics were dried (MgSO₄), filtered through silica, eluted with methylene chloride, and concentrated to an orange solid II (16.8 g, 75% yield).

### Preparation of tert-butyl 2,6-difluorophenylcarbamate (III)

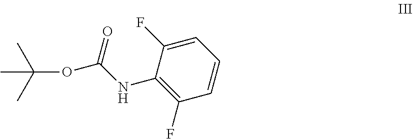

Boc-2,6-difluoroaniline (4.5 mL, 42 mmol, 1.0 equiv.) and Boc anhydride (11.1 g, 51 mmol, 1.2 equiv.) were mixed in THF and to this mixture was added 1M NaHMDS (100 mL, 100 mmol, 2.3 equiv.) at rt. HPLC-MS confirmed the formation of the desired product [M+1]=230. Added 50 mL of brine, evaporated off the THF and extracted into EtOAc (2×100 mL). The combined organics were washed with brine (1×50 mL), followed by citric acid (2×10%). The resulting solution was dried over MgSO₄ anh., filtered and concentrated the filtrate to furnish an orange solid that was used directly in the next step without additional purification. Retention time on HPLC was 15 min.

Step A: Preparation of 2-(2,4-Difluorophenyl)-6-(2,6-difluorophenylamino)-nicotinic acid ethyl ester (IV)

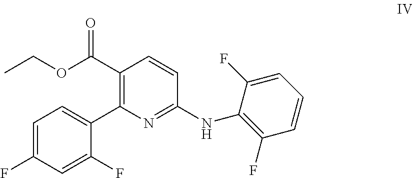

### Method A:

To a nitrogen purged flask was charged palladium acetate (13.2 g, 59 mmoles, 0.04 eq), racemic BINAP (36.6 g, 59 mmoles, 0.04 eq), followed by 1.9 L toluene. The heterogeneous slurry was heated to 50° C. under nitrogen for 2 hours, cooled to 30° C., then the pyridyl chloride II (386.4 g, 1.45 moles, 1.0 eq) and Boc-2,6-difluoroaniline III (386.4 g, 1.69 moles, 1.2 eq), and K₃PO₄ (872 g, 4.1 moles, 2.8 eq) were added all at once followed by a 1.9 L toluene rinse. The heterogeneous reaction mixture was heated to 100° C. overnight and monitored by HPLC. When the reaction showed complete conversion to 43 by HPLC [Tᵣₑₜ II=17 min, Tᵣₑₜ 43=20.5 min, Tᵣₑₜ IV=17.6 min, monitored at 229 nm] (usually between 18-20 hours) the reaction was cooled to room temperature and the contents diluted with 1.94 L EtOAc. To this was added 1×1.94 L of 6N HCl, and both layers were filtered through celite. The celite wet cake was rinsed with 2×1.9 L EtOAc. The layers were separated and the organic layer washed with 1×1.9 L of brine, dried (MgSO₄), filtered and concentrated to a brown, viscous oil. To remove the Boc-protecting group, the oil was dissolved in 1.94 L of methylene chloride and 388 mL TFA was added. The reaction was stirred overnight to facilitate Boc removal. The volatiles were removed in vacuo, EtOAc (1.9 L) and sufficient quantity of 1 or 6 N NaOH was added until the pH was 2-7. Then a sufficient quantity of 5% NaHCO₃ was added to bring the pH to 8-9. The organic layer was separated and washed with 1×5% NaHCO₃, dried (MgSO₄), filtered an concentrated to a brown oil/liquid. The crude oil/liquid was azeodried twice with a sufficient quantity of toluene. At times the free base precipitated out resulting in a slurry. The residue was dissolved in 500 mL toluene and 1.6 L 1N HCl/ether solution was added, which resulted in the solid HCl salt crashing out. Heat was applied until the homogenized/solids broke up. If necessary, 200 mL of EtOAc can be added to facilitate the break up. After cooling, the solid IV was isolated by vacuum filtration and re-crystallized from EtOH. Yields for these two consecutive steps usually ranged between 50-70%.

### Method B.

In a 1 L, 4-necked, round-bottomed flask equipped with an overhead mechanical stirrer, heating mantle, reflux condenser, and thermocouple was charged II (50 g), Cs₂CO₃ (150 g) and 0.15 L of NMP. The solution was stirred vigorously and heated to 65° C. at which time to the suspension was added a solution of III (60 g) in 0.10 L of NMP over 10 minutes. Heating at 65° C. for 18 hours, HPLC showed ˜85% conversion of II to the desired Boc adduct. At this time, the temperature was increased to 75° C., and HPLC analysis after heating for an additional 18 hours showed ˜97% conversion of II to the desired Boc adduct Boc-IV (not shown). The mixture was then cooled to 20° C. and poured in one portion into 2.0 L of water, stirring in a 4-necked, 3 L, round-bottomed flask equipped with an overhead mechanical stirrer and thermocouple. The temperature of the water rose from 22° C. to 27° C. as a result of the addition of the NMP solution. The suspension was then cooled to 15° C. and the tan solid was collected by filtration, rinsed with water and pulled dry on the filter for 2 hours. Then, in a 2 L, 4-necked, round-bottomed flask equipped with an overhead mechanical stirrer and thermocouple was charged the tan solid and 0.8 L of CH₂Cl₂. To the stirred solution was added 70 mL of TFA in one portion. After two hours stirring at ambient temperature, none of the Boc protected material was detected by HPLC, and the mixture was concentrated by rotary evaporation. The oily residue was taken up in 0.7 L EtOAc, and treated with 0.7 L saturated NaHCO₃, during which gas was produced. The EtOAc layer was washed with 0.25 L saturated NaCl and concentrated by rotary evaporation. To the resultant brown oil was added 0.2 L EtOAc and the solution treated with HCl in Et₂O (0.4 L of 2.0 M solution) and stirred for 60 minutes. The product IV-HCl, a yellow powder, was collected by filtration. The product may be recrystallized by heating the crude salt in 4 mL EtOH/g of crude product to reflux, then cooling to ambient temperature (70.5% yield).

### Step B: 6-1-(2,6-Difluorophenyl)-2-(2,4-difluorophenyl)-nicotinic acid (V)

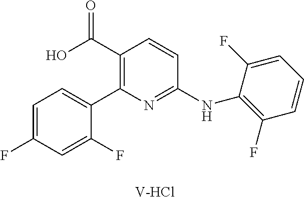

Water (590 Kg) was charged into a 1900 L reactor. With agitation, hydrochloric acid (37%, 804 Kg) was charged, followed by an additional 174 Kg of water. Finally ester IV-HCl (90.7 Kg, 213 moles) was charged followed by THF. The mixture was heated to 95-100° C. for 36 hours. At this point, TLC of an aliquot worked up by a simple water washing (Silica gel, F₂₅₄; 3.0×6.5 cm; 1:4 acetone:hexanes, IV—R_{f}=0.3, V—R_{f}=0.2) indicated completion of the reaction. This was confirmed by HPLC. After 36 h, the reaction temperature was allowed to reduce down to 22° C. and the resulting mixture agitated at this temperature for 3-4 h. The resulting precipitate was collected by filtration. The filtration cake was washed with water until the pH of the filtrate was 3-4 by wet pH paper (usually 5 washings). The solid was then dissolved in THF/water/HCl (1300 Kg/84 Kg/199 Kg) and treated with charcoal (10 Kg) to remove impurities. After filtration, washing with water and drying under vacuum, product V-HCl was obtained as a white-yellow solid (211 Kg, 78% yield).

### Step C: 6-1-(2,6-Difluoro-phenyl)-ureido]-2-(4-fluoro-phenyl)-nicotinic acid (I)

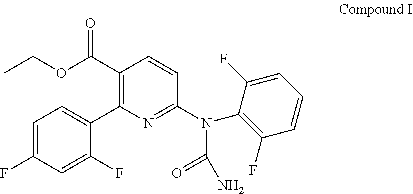

To a nitrogen purged flask was charged the amino ester HCl salt of IV (262 g, 0.67 mole, 1.0 eq), followed by 1.2 L toluene. To the heterogeneous mixture was added phosgene (1.4 L of 1.93 M toluene solution, 2.7 moles, 4.0 eq) and the reaction was heated to 50° C. under nitrogen overnight. The progress of the reaction to form the —NC(O)Cl moiety was monitored by HPLC [Tᵣₑₜ IV=17.6 min, Tᵣₑₜ carbamoyl intermediate=19.7 min, Fᵣₑₜ I=16.4 min, monitored at 229 nm]. Once the nitrogen was completely reacted, the brown solution was cooled to approximately −5° C., and NH₄OH (0.84 L, 12.4 moles, 18.5 eq) was slowly added dropwise. As the addition neared completion a solid formed. The slurry was stirred with 1 L of water and collected by vacuum filtration. The wet cake was washed with 1×390 mL toluene to remove late eluting impurities. The product was further purified by crystallization in MeOH giving Compound I as a white solid.

Compound I can also be synthesized as described below.

### Preparation of ethyl 6-chloro-2-(2,4-difluorophenyl)nicotinate (5)

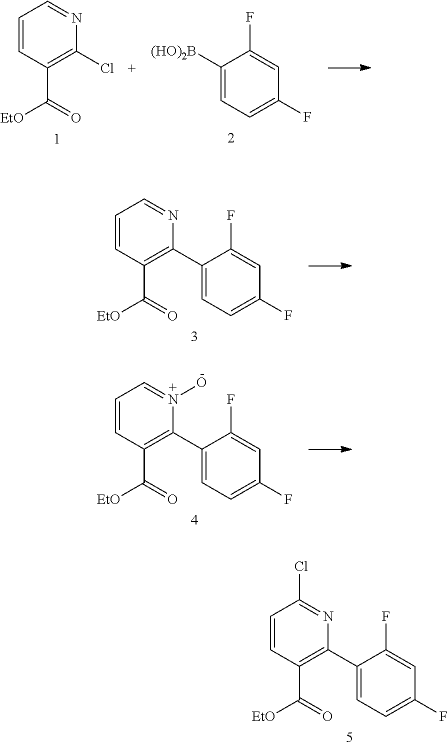

### Preparation of ethyl 2-(2,4-difluorophenyl)nicotinate (3)

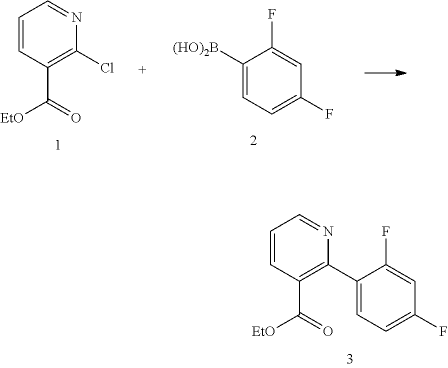

To a nitrogen purged 3.0 L, 4-necked flask, fitted with an overhead stirrer, thermocouple, heating mantle, nitrogen outlet and reflux condenser, was charged Pd(Ph₃)₄ (5.0 g, 4.33 mmoles, 0.005 eq), sodium carbonate (92.6 g, 874 mmoles, 1.3 eq), ethyl 2-chloronicotinate, 1 (126.0 g, 678 moles, 1.0 eq), 2,4-difluorophenylboronic acid, 2 (125 g, 791 mmoles, 1.2 eq), followed by 0.5 L of toluene and 125 mL denatured EtOH. The reaction was heated to 82° C. with vigorous stirring under N₂ overnight (completeness of reaction determined by HPLC and TLC). The reaction was cooled to room temperature, the mixture filtered through a small pad of Celite® and the solvents removed under vacuum at 55° C. The residue was dissolved in EtOAc, washed, dried (MgSO₄), filtered through Celite® again, and concentrated. The product was obtained as a yellow solid.

### Preparation of 2-(2,4-difluorophenyl)-3-(ethoxycarbonyl)pyridine 1-oxide (4)

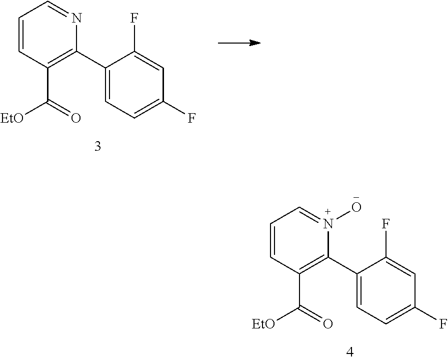

To a nitrogen purged, 12 L, 5-necked flask, fitted with an overhead stirrer, a thermocouple and a condenser, was charged ethyl 2-(2,4-difluorophenyl)nicotinate, 3 (144 g, 548 mmoles, 1.0 eq), and 4 L of CH₂Cl₂. With stirring, mCPBA was added over 5 minutes, and the temperature was gradually increased from 22 to 38° C. in 45 minutes (completeness of reaction determined by HPLC). The reaction was cooled to room temperature and the contents slowly poured into 3 L of water. Na₂SO₃ was added slowly (exotherm from 20 to 33° C.) until the peroxide test (starch/I₂ paper) indicated no peroxides remained in the mixture. The aqueous layer was separated and the organic layer was washed with saturated NaHCO₃ (˜3 L). The organic layer was dried with MgSO₄, filtered, and concentrated to a brown thick oil. The oil was then treated with MTBE (2 L) and stirred to give a white precipitate, which was collected by filtration, washed with MTBE and dried under vacuum to give the title compound 4.

### Preparation of ethyl 6-chloro-2-(2,4-difluorophenyl)nicotinate (5)

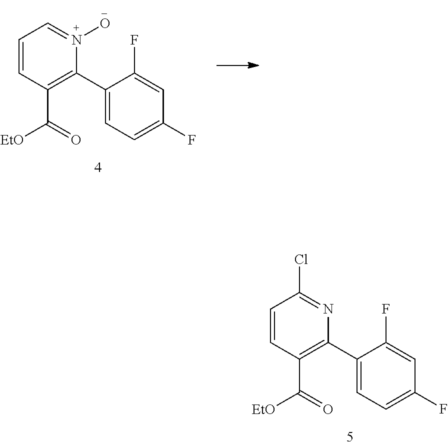

To a nitrogen purged 500 mL, 3-necked flask, fitted with a reflux condenser, heating mantle and a thermocouple was charged 2-(2,4-difluorophenyl)-3-(ethoxycarbonyl)pyridine 1-oxide, 4 (21 g, 75 mmoles, 1.0 eq), followed by 150 mL dichloroethane. Phosphorous oxychloride (75 mL) was added in one aliquate with stirring, causing an immediate rise in temperature from 21 to 23° C. followed by gradual warming. The solution was heated under nitrogen to 70-75° C. (completeness of reaction determined by HPLC). The reaction was then cooled to room temperature and concentrated under vacuum to remove most of the POCl₃. The remainder was quenched by slowly pouring onto 450 g of ice. The mixture (after the ice melted) was then extracted into methylene chloride (2×200 mL). The combined organics were dried (MgSO₄), filtered through silica, eluted with methylene chloride, and concentrated to give the title compound, 5, as an orange solid. H NMR (500.0 MHz, CDCl₃) d 8.15 (d, J=8.2 Hz, 1H), 7.54 (td, J=8.5, 5.0 Hz, 1H), 7.34 (d, J=8.2 Hz, 1H), 6.96-6.92 (m, 1H), 6.79-6.74 (m, 1H), 4.16 (q, J=7.2 Hz, 2H), 1.10 (t, J=7.1 Hz, H) ppm.

### Preparation of tert-butyl 2,6-difluorophenylcarbamate (7)

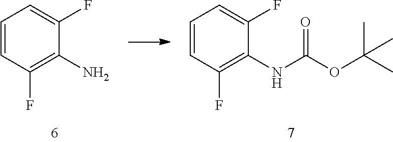

2,6-Difluoroaniline, 6 (4.5 mL, 42 mmol, 1.0 equiv.), and Boc anhydride (11.1 g, 51 mmol, 1.2 equiv.) were mixed in THF and to this mixture was added 1M sodium hexamethyldisilazide (100 mL, 100 mmol, 2.3 equiv.) at room temperature (completeness of reaction determined by HPLC). 50 mL brine was then added, and the solution was concentrated and extracted with EtOAc (2×100 mL). The combined organics were washed with brine (1×50 mL), followed by citric acid (2×10%). The resulting solution was then dried over MgSO₄, filtered and concentrated to give the title compound, 7, as an orange solid which was used directly in the next step without additional purification. H NMR (500.0 MHz, CDCl3) 7.18-7.13 (m, 1H), 6.96-6.91 (m, 2H), 6.06 (s, 1H) and 1.52 (s, 9H) ppm

### Preparation of ethyl 6-(tert-butoxycarbonyl(2,6-difluorophenyl)amino)-2-(2,4-difluorophenyl)nicotinate (8)

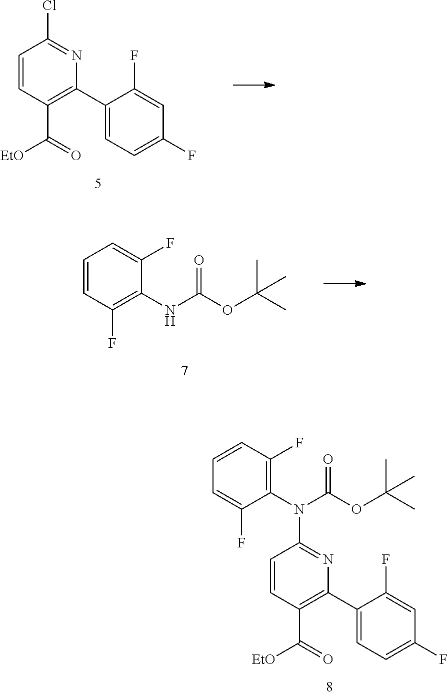

A mixture of compound 5 (100.82 g, 0.33 mol, 1.0 equiv.), compound 7 (101.05 g, 0.44 mol, 1.30 eq), and cesium carbonate (177.12 g, 0.54 mol, 1.60 eq) was suspended in DMSO (250 mL, 2.5 volumes) and stirred vigorously at 55-60° C. for 48 hours (completeness of reaction determined by HPLC). The mixture was cooled to 20-30° C. and the base was quenched by careful and slow addition of a 1 N HCl (aq) solution (540 mL, 1.60 eq), keeping the internal temperature of the reaction mixture below 30° C. Upon cooling, a precipitate formed and was filtered and washed with water (2×250 mL, 2×2.5 volumes). The filtrand was suspended in absolute ethanol (1000 mL, 10 volumes) and heated to reflux. The reflux was maintained for 30-60 minutes, and water (200 mL, 2 volumes) was added to the mixture. The resulting mixture was then heated again to reflux, and reflux was maintained for 30 minutes, at which point the suspension was cooled to 10° C. The resulting solids were then filtered and washed with water (2×250 mL, 2×2.5 volumes), followed by absolute ethanol (250 mL, 2.5 volumes), and then transferred to a vacuum oven and dried at 50-60° C. The title compound, 8, was obtained as a white crystalline solid. (¹H NMR, 500 MHz; CDCl₃) δ 8.28 (d, 1H), 8.12 (d, 1H), 7.19 (q, 1H), 6.96 (t, 2H), 6.81 (t, 1H), 6.74 (t, 1H), 4.25 (q, 2H), 1.50 (s, 9H), 1.20 (t, 3H).

### Preparation of 2-(2,4-difluorophenyl)-6-(2,6-difluorophenylamino)nicotinic acid (9)

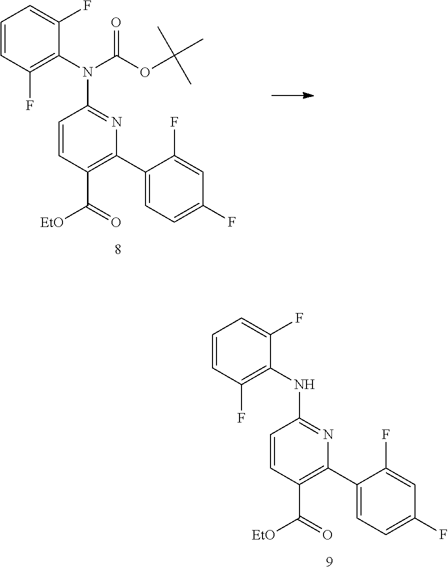

To compound 8 (100 g, 0.204 mol, 1.00 eq) was added a 7M sulfuric acid solution prepared by the slow addition of concentrated sulfuric acid (285 mL, 2.85 vol, 5.24 mol) to distilled water (465 mL, 4.65 vol) while keeping the temperature below 50° C. The mixture was heated at 100±5° C. until the reaction was complete. The mixture was then cooled to 30±5° C. and additional water (750 mL, 7.5 vol) was added. Isopropyl acetate (2 L, 20 vol) was then added and the mixture was stirred for 15 minutes. Stirring was stopped and the phases were allowed to separate. The aqueous phase was separated and water (7.5 vol) was charged to the organic phase. The mixture was stirred for 15 minutes, polish filtered, then the aqueous phase was drained. The total volume of the organic layer was reduced to 4 vol by vacuum distillation at 45±5° C. The resulting slurry was cooled to −10° C. for 12 hours and filtered. The filtrand was washed with cold isopropyl acetate (3 vol) and the solids were dried under vacuum at 50±5° C. to give the title compound, 9, as a white solid. (¹H NMR, 500 MHz; DMSO-d₆) δ 12.50 (s, 1H), 9.25 (s, 1H), 8.07 (d, 1H), 7.39 (q, 1H), 7.29 (m, 1H), 7.18 (m, 3H), 7.09 (m, 1H), 6.25 (m, 1H).

### Preparation of 2-(2,4-difluorophenyl)-6-(1-(2,6-difluorophenyl)ureido)nicotinamide (10)

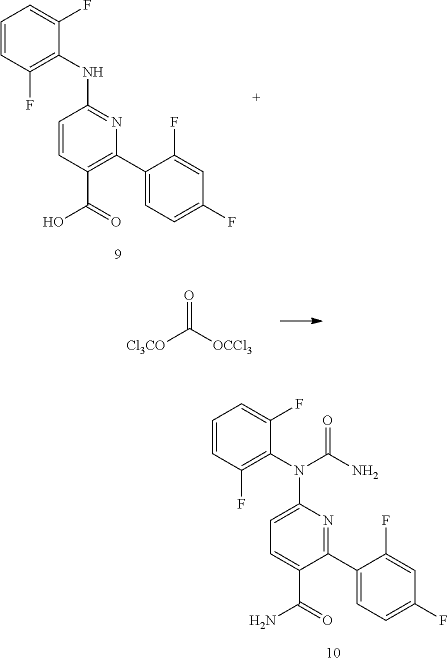

Triphosgene (38.87 g, 0.1276 mol, 0.9 eq) and compound 9 (51.14 g, 0.1412 mo, 1 eq.) were charged to a reactor. Anhydrous THF (486 mL, 9.5 vol) was then added and the clear solution was cooled to −30±5° C. Diisopropylethylamine (73.79 mL, 0.424 mol, 3.0 eq) in THF (103 mL, 2.5 vol) was charged to the reactor keeping the temperature below −20° C. After addition, the reaction mixture was warmed to 20±3° C. The mixture was stirred for 2 hours and was then filtered through Celite®, and the cake was rinsed with THF (767 mL, 15 vol). The filtrate was cooled to −30° C. and anhydrous NH₃ (3 equiv.) added. The resulting white slurry was purged with N₂ and warmed up to 20±3° C. for 1 hour. The reaction mixture was then cooled to 0±5° C. for 30 minutes. The mixture was again filtered and the reactor was rinsed with THF (255 mL, 5 vol). The cake was rinsed with H₂O (255 mL, 5.0 vol) followed by 1N H₂SO₄ (10 vol). The solid was then transferred to a vacuum oven and dried at 35±3° C. to give the title compound, 10, as a white solid. (¹H NMR, 500 MHz; DMSO-d₆) δ 7.97 (d, 1H), 7.85 (s, 1H), 7.56 (quin, 1H), 7.45 (q, 1H), 7.40 (s, 2H), 7.28 (t, 3H), 7.15 (td, 1H), 7.06 (d, 1H).

### Preparation of a solid form of 2-(2,4-difluorophenyl)-6-(1-(2,6-difluorophenyl)ureido)nicotinamide (10)

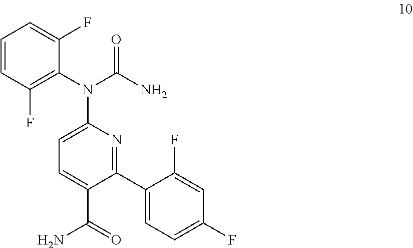

A slurry of compound 10 (407.74 mL, 1.01 mol, 1.00 eq) in methanol (6.52 L, 16.0 vol) was heated to 60° C. until a solution was obtained. The reactor contents were then cooled to 48° C., held at this temperature until crystallization set in, stirred for 30 minutes and then cooled to 0° C. The slurry was filtered off, the reactor and filter cake were rinsed with methanol (816 mL, 2 vol) previously cooled to 0-5° C. The filter cake was dried under vacuum for 30 minutes. The solid was then returned to the reactor and stirred with a 1:3 methanol:water mixture (4.1 L, 10 vol) at 22° C. for 24 hours. Methanol (2.05 L, 5 vol) was added to the reactor, resulting in a 1:1 methanol:water mixture. This solution was then stirred for an additional 24 hours, after which the mixture was filtered, and the cake was rinsed with water (818 L, 2 vol). The solids were transferred to a vacuum oven and dried at 38° C. to give compound 10 as a white solid.

### Alternative Route to 2-(2,4-difluorophenyl)-6-(2,6-difluorophenylamino)nicotinic acid (9)

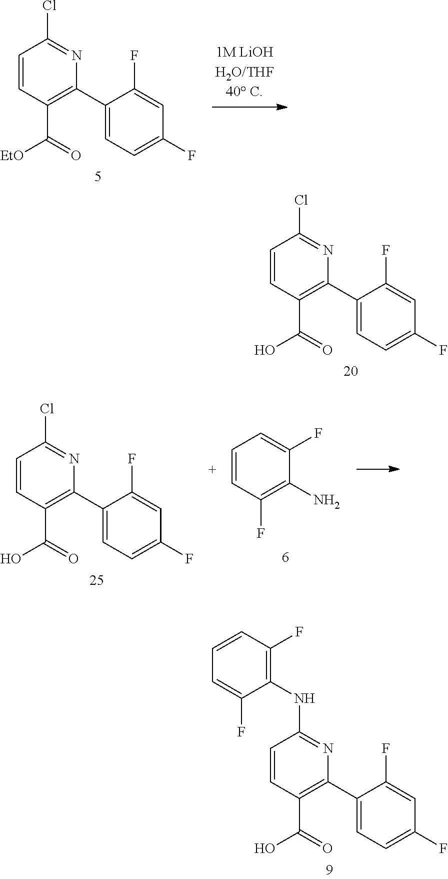

### Step A: Saponification:

A 250 mL round bottom flask was charged with Compound 5 and THF at room temperature. A 1M LiOH solution was then added to flask. The resulting mixture was heated to approximately 40° C. for about 3 hours and then cooled down room temperature and stirred for about 2 days. The reaction can be monitored by HPLC. After stirring, the mixture is transferred washed with 100 mL water and 100 mL DCM. The organic layer was separated and neutralized with 110 mL aqueous 1N HCl. The aqueous layer was extracted with DCM (3×100 mL). The organic layers were combined and concentrated to provide a white solid Compound 20. H NMR (500.0 MHz, DMSO) 13.5 (bs, OH) d 8.31 (d, J=8.3 Hz, H), 7.70 (d, J=8.2 Hz, H), 7.62 (dd, J=8.6, 15.2 Hz, H), 7.35-7.31 (m, H), 7.21 (td, J=8.5, 3.6 Hz, H), 3.33 (s, H), 2.51 (d, J=1.7 Hz, H) ppm.

### Step B: Coupling

A 100 mL round bottom flask was charged with Compound 20 (1.0015 g, 3.714 mmol) in MBTE (10 mL) followed by the addition of Compound 6 (600 μL, 5.572 mmol). The resulting mixture was cooled to an internal temperature of −8° C. to −10° C. with an ice/acetone bath followed by the dropwise addition (over 1 hour) of a 1 M solution of potassium bis(trimethylsilyl)amide (9.3 mL, 9.300 mmol) while maintaining the mixture temperature at less than about −5° C. After the addition of the base, the reaction mixture was quenched with 20 mL 1 M HCl at room temperature. The mixture was washed with 20 mL water and 50 mL ethyl acetate. The aqueous phase was washed at least once more with ethyl acetate. The organic layer was concentrated followed by the addition of DCM (25 mL). The resulting solids were suspended, filtered, and washed with 50 mL DCM. Analysis of the solids confirmed the presence of Compound 9.

In other embodiments the base used in the coupling step can also be selected from LiHMDS (55° C.), NaHMDS (55° C.), KOtBu, and nBuLi.

### Alternative Route to 2-(2,4-difluorophenyl)-6-(1-(2,6-difluorophenyl)ureido)nicotinamide (10)

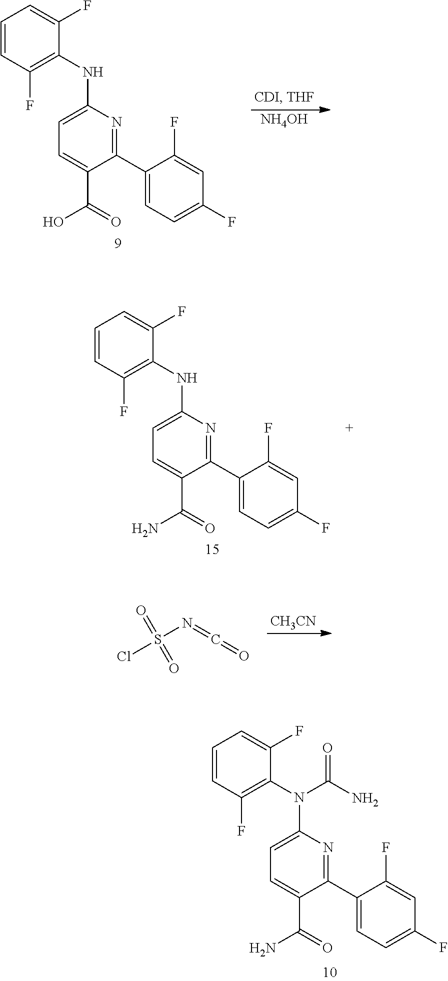

In some embodiments, Compound 10 can be produced by stepwise formation of amide Compound 15 using CDI, THF, NH₄OH or toluene/Methylchloroformate/NEt₃/NH₄OH. Compound 10 can be subsequently formed by treating Compound 15 with chlorosulfonylisocyanate in a solvent such as CH₃CN, DMSO, MeTHF, THF, DMF, or DMSO.

### Detailed Experimental Procedures

### Example I

### Preparation and Physical Characterization of Form C

Form C was prepared using the following procedure:

Precursor methanol solvate Form A (prepared as described in Example IV below) was dissolved in 20 volumes of a 1:3 MeOH:H₂O mixture at 0° C. for 24 h. More methanol was then added to achieve a ratio of MeOH:H₂O 1:1. The ppt formed was collected by filtration and air-dried. A characteristic ¹H NMR spectrum for Form C is depicted in FIG. 2.

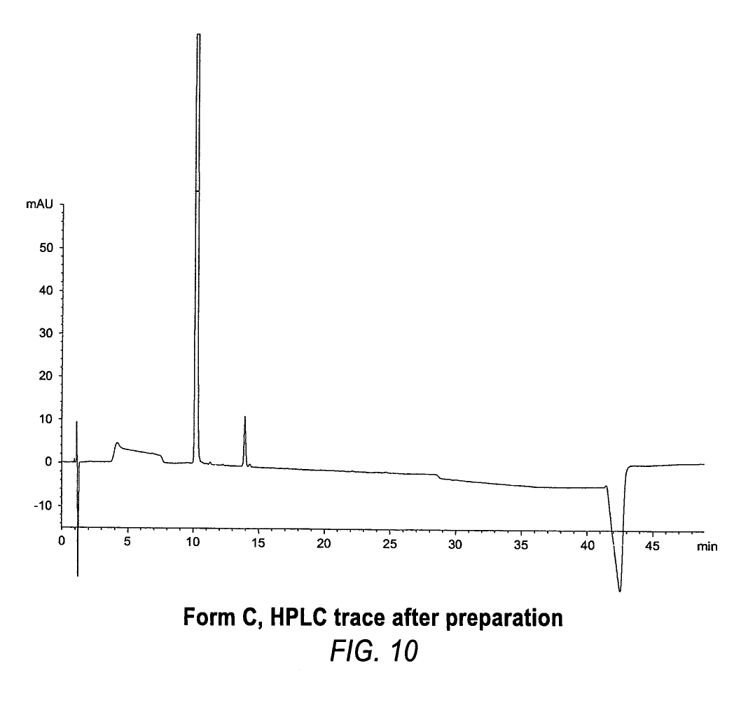

Form C was characterized as a white powder consisting of particles <10 mm in size with no discernible morphology. Purity analysis was carried out using HPLC and showed this powder to be 98.8% pure, with traces of decomposition product Y. (HPLC trace FIG. 10).

Table XIII contains representative apparent (non-equilibrium) solubilities of Form C in various exemplary solvents. This data was obtained by using the following general procedure:

Fifty (50.0) mg of Compound I, Form C was weighed into a small, screw-top vial. The relevant solvent was added in portions until a clear solution was obtained. In several cases, solubilization was aided by heating with a heat gun and then allowed to cool down to rt. The data provided in Table XIII are representative and some variability may occur between lots of a particular sample, e.g., about 10%.

TABLE XIII
Apparent solubilities for Form C in various solvents.

| Solvent | Solubility mg/mL |
|---|---|
| Acetonitrile | 33 |
| Acetone | 50 |
| 1-Butanol | 50 |
| 2-Butanone | 50 |
| Dimethylformamide | 100 |
| Ethanol | 50 |
| Ethyl Acetate | 20 |
| Heptane | <20 |
| Methanol | 100 |
| Methylene Chloride | <20 |
| 2-Propanol | 50 |
| 2-Propyl Acetate | <20 |
| N,N-dimethylacetamide | 100 |
| cyclohexane | <20 |
| 1,4-dioxane | 100 |
| Ethyl acetate | 20 |
| Butyl acetate | 33 |
| Methyl isobutyl ketone | 50 |
| Tetrahydrofuran | 20 |
| Toluene | 20 |
| Tert-butyl methyl ether | <20 |
| DMSO | 100 |
| Hexafluorophenol | 100 |
| Water | <20 |

Form C was additionally characterized by using several physical characterization techniques described herein

An exemplary XRPD trace for Form C is displayed in FIG. 1. A single crystal of Form C, suitable for single crystal X-Ray crystallography was obtained by slow re-crystallization of Form A from EtOAc using the crystallization procedure M (maturation of Form C) described in the foregoing section.

A schematic of the crystal packing is depicted in FIG. 6. The molecules in this form are seen to dimerize forming hydrogen bonds between the ureido (H₂NOCNH—) and aminocarbonyl (—OCNHR) groups. Form C has a space group C_{c}, having the following unit cell dimensions: a=10.9241 Å, b=24.2039 Å, c=7.0124 Å, α=90°, β=111.0685°, γ=90°, δ_{calc} (g/cm³)=1.552.

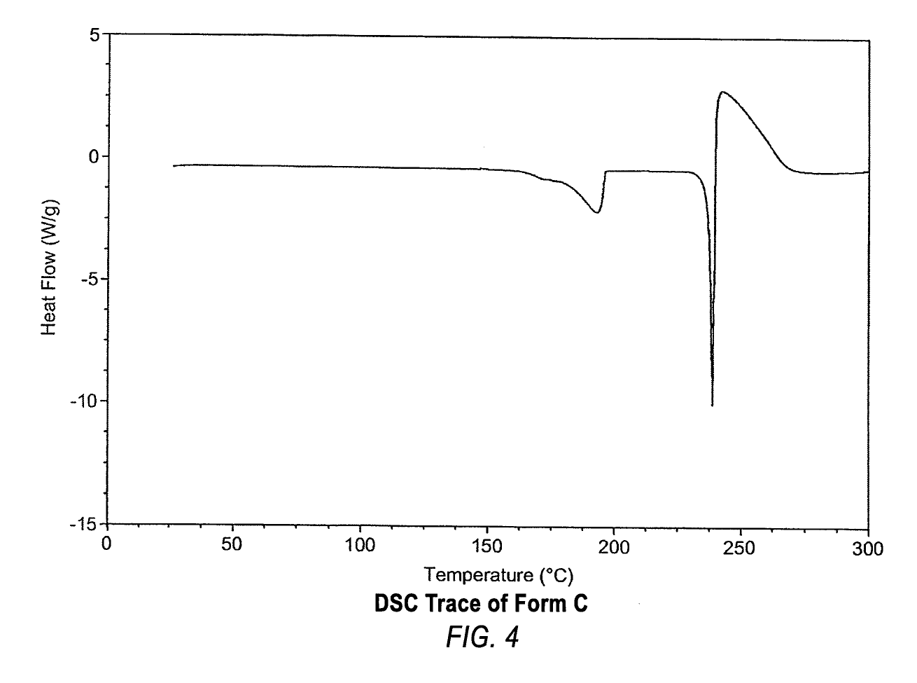

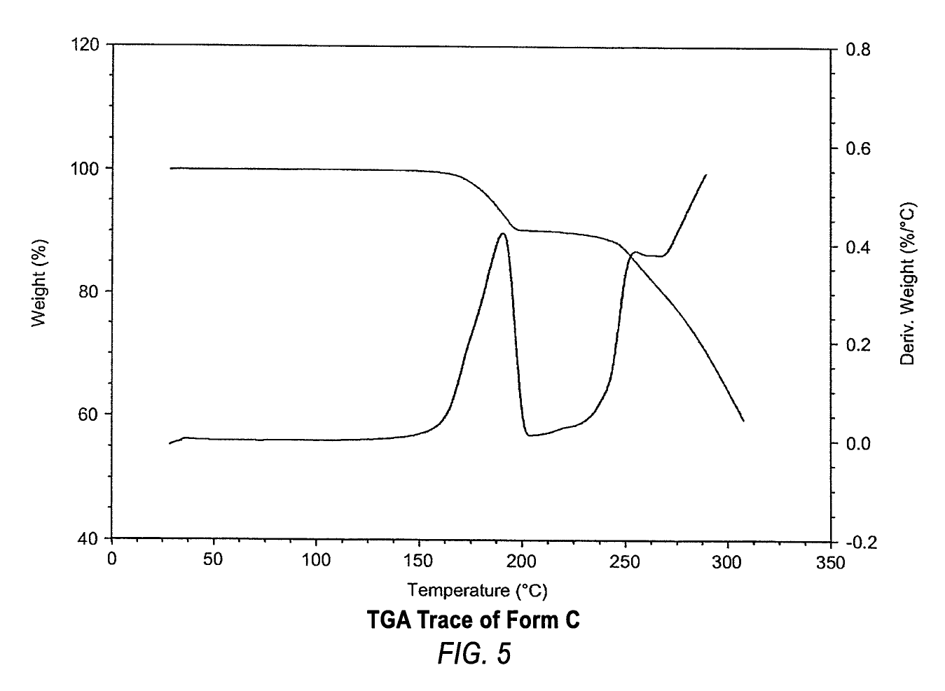

A characteristic DSC thermogram and a characteristic TGA thermogram for Form C are shown in FIG. 4 and FIG. 5, respectively. An endotherm beginning at 178° C., that plateaus slightly and then peaks at 193° C. is measured in DSC. Further, this endotherm coincides with a 9.5-10.5% weight loss measured by TGA.

A characteristic FT-IR spectrum for Form C is depicted in FIG. 3.

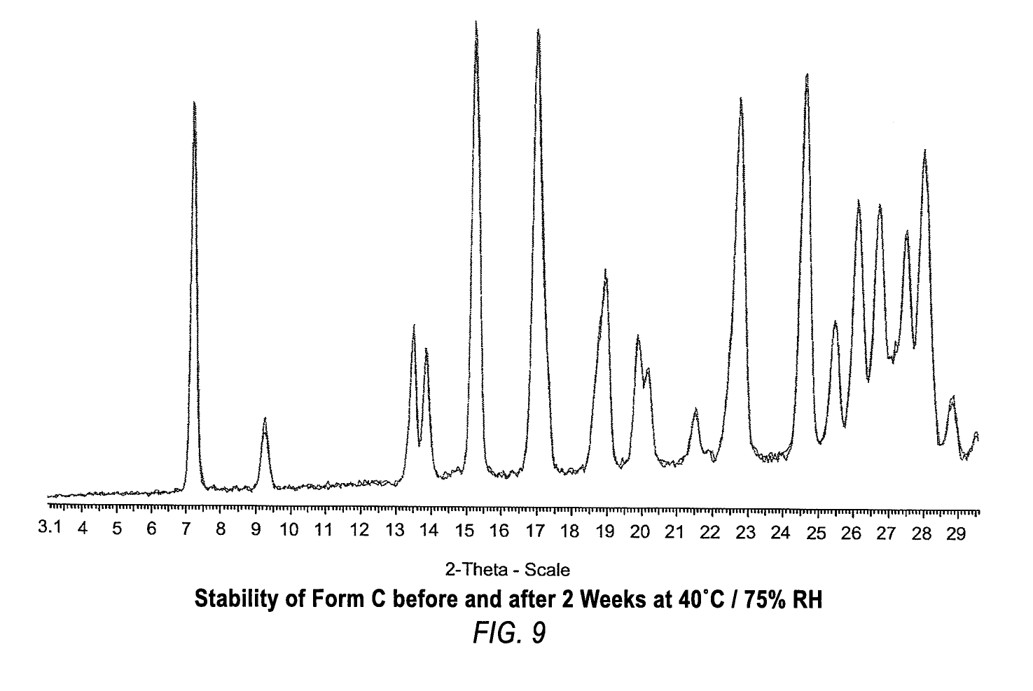

In stability studies, Form C was found to remain substantially in the same physical and chemical form for at least two weeks at 40° C./75% RH (see FIG. 9).

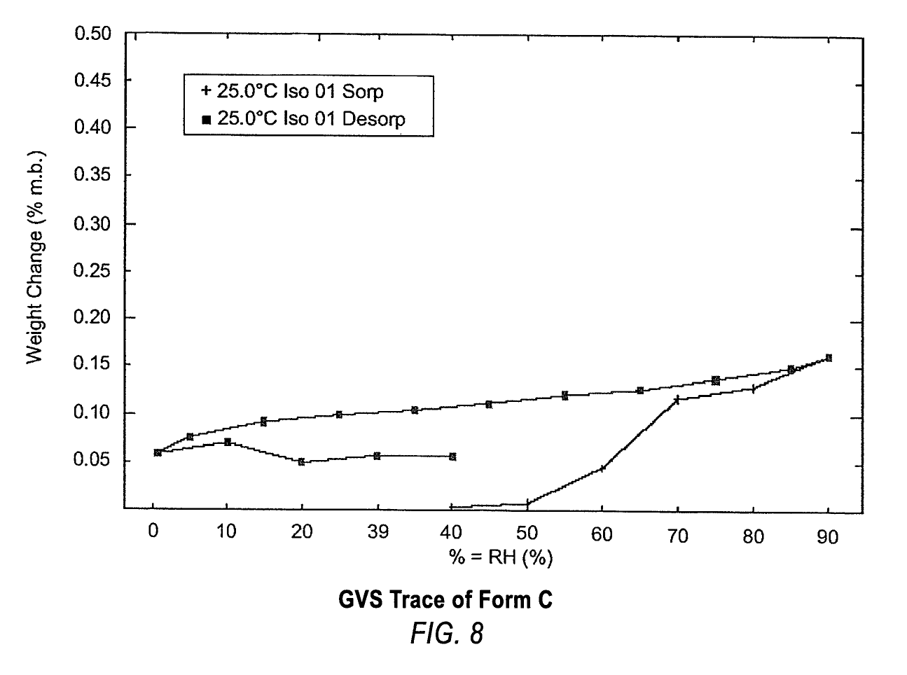

On analysis of its GVS trace (FIG. 8), Form C displayed a negligible weight gain up to 60% RH and a low total weight gain of 0.15% from 0 to 90% RH at T=25° C.

### Example II

### Preparation and Physical Characterization of Form F

Form F was prepared using the following procedure:

60 mg of Compound I, Form C, was slurried in 10 mL of ethyl acetate for 30 minutes with stirring. The slurry was then filtered through a 0.45 μm PTFE filter. The filtrate was triturated into 50 mL of hexanes pre-cooled to −20° C. Precipitation occurred instantly. After 2 hours at −20° C., the solid was isolated by filtration, air dried and analyzed by XRPD. The sample was obtained as a white solid, which was partially crystalline with no discernible morphology. The sample also contained trace amounts of Form C. A typical ¹H NMR spectrum for Form F is depicted in FIG. 12.

Form F was additionally characterized by using several physical characterization techniques described herein.

A representative XRPD pattern of Form F is provided in FIG. 11. Suitable crystals for single crystal X-Ray crystallography were not obtained.

An exemplary FT-IR spectrum for Form F is depicted in FIG. 13. Representative peaks in this IR are: NH stretch at 3494 nm, CO and NH bend region peaks at 1720, 1700, 1678 nm.

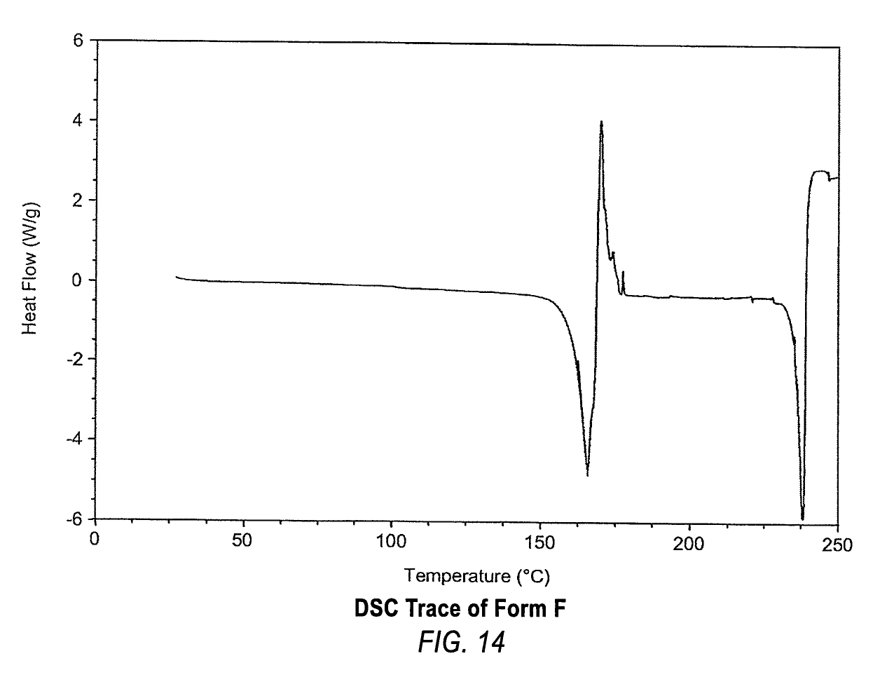

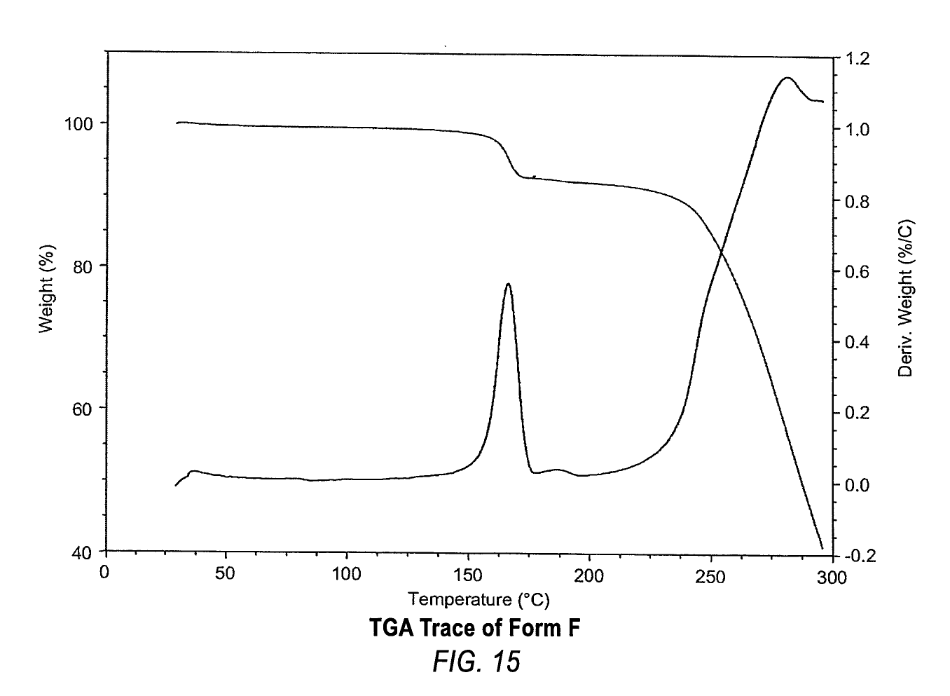

Characteristic DSC and TGA traces for Form F are displayed in FIG. 14 and FIG. 15, respectively. According to these, Form F is characterized by an endothermal event beginning at 160° C. and peaking at 165° C. as measured in DSC. Further, this thermal event coincides with a 6.8% net weight loss between 130° C. and 180° C. as measured by TGA and due to degradation.

Form F displayed solubility in water of at least 0.021 mg/mL at 25° C.

In stability studies, Form F remained in substantially the same physical form for at least 2 weeks at 40° C./75% RH. Further, Form F remained chemically stable for at least 2 weeks at 40° C./75% RH.

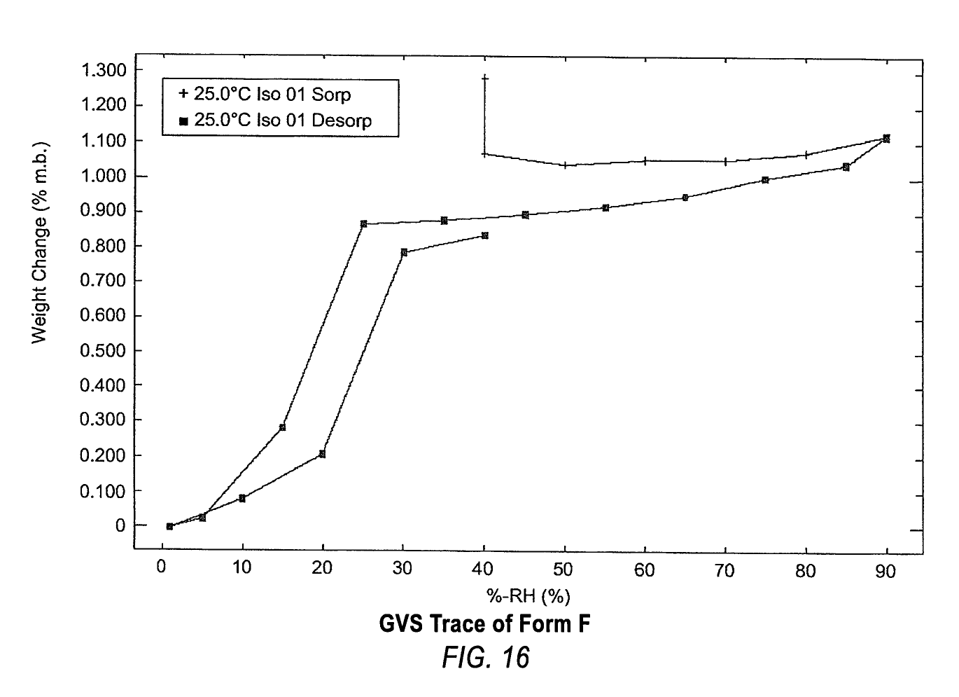

Form F displays a total weight gain in water of 1% at 40% RH and a maximum of 1.1% at 90% RH as seen by GVS (FIG. 16).

### Example III

### Preparation and Physical Characterization of Form G

Form G was prepared using the following procedure:

240 mg of Compound I, Form C, was slurried in 40 mL of ethyl acetate for 30 minutes with stirring. The slurry was then filtered through a 0.45 μm PTFE filter. The filtrate was triturated into 50 mL of hexanes pre-cooled to −20° C. Precipitation occurred instantly. After 24 hours at −20° C., the solid was isolated by filtration, air dried and analyzed by XRPD. The sample was additionally vacuum dried at 30° C. for 48 h. Form G is characterized by a ¹H NMR spectrum as depicted in FIG. 18. Form G was prepared as a white solid and was seen to be crystalline with no discernible morphology. Upon exposure to moisture, Form G becomes its hydrate Form Q.

A representative XRPD pattern of Form G is provided in FIG. 17. Crystals of enough quality for single crystal X-Ray analysis could not be obtained.

Form G can be characterized by a FT-IR spectrum as depicted in FIG. 19.

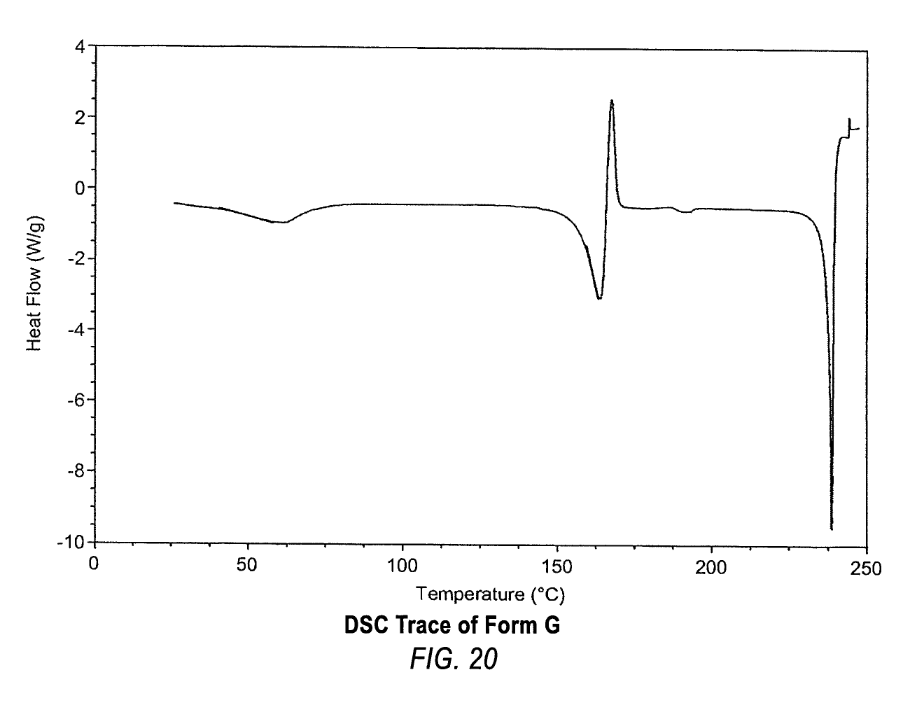

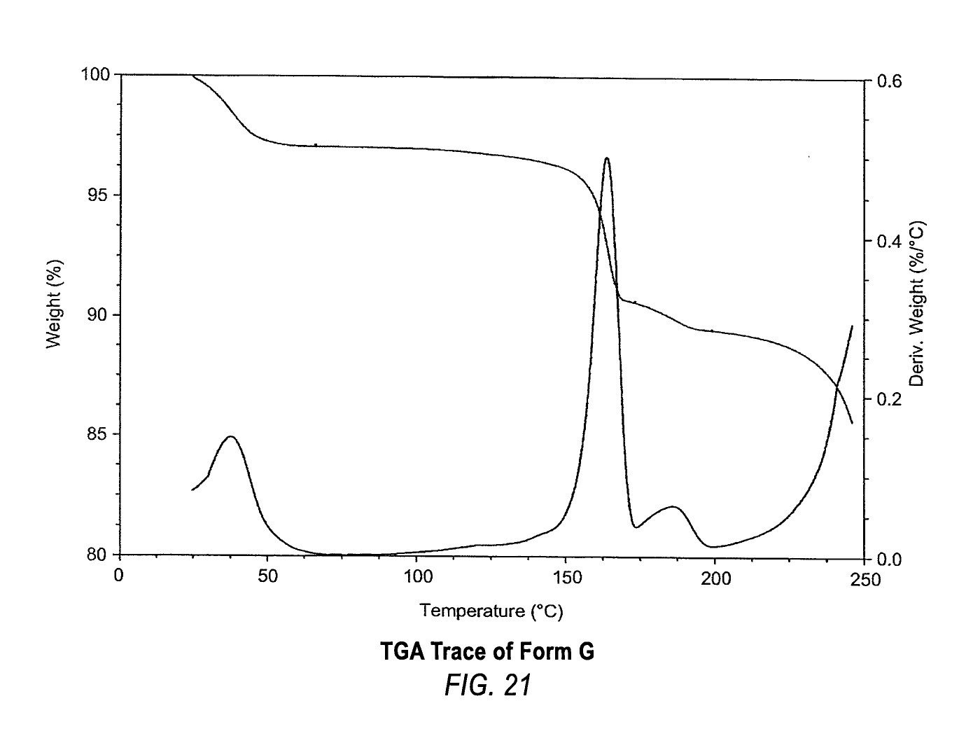

Representative DSC and TGA traces are displayed in FIG. 20 and FIG. 21, respectfully. According to these, Form G can be further characterized by an endothermal event beginning at 156° C. and peaking at 163° C. as measured by DSC. Further, this coincides with a 6.5% net weight loss between 95° C. and 175° C. as measured by TGA and can be attributed to a decomposition event. Form G can further be characterized by a second endotherm beginning at 36° C. and peaking at 61° C. as measured in the DSC. This corresponds to a 2.9% net weight loss between 25° C. and 70° C. as measured by TGA.

Form G displays solubility in water of at least 0.020 mg/mL at 25° C.

In stability studies, Form G remained in substantially the same physical form for at least two weeks at 40° C./75% RH. Further, Form G remained chemically stable for at least two weeks at 40° C./75% RH (see FIG. 26).

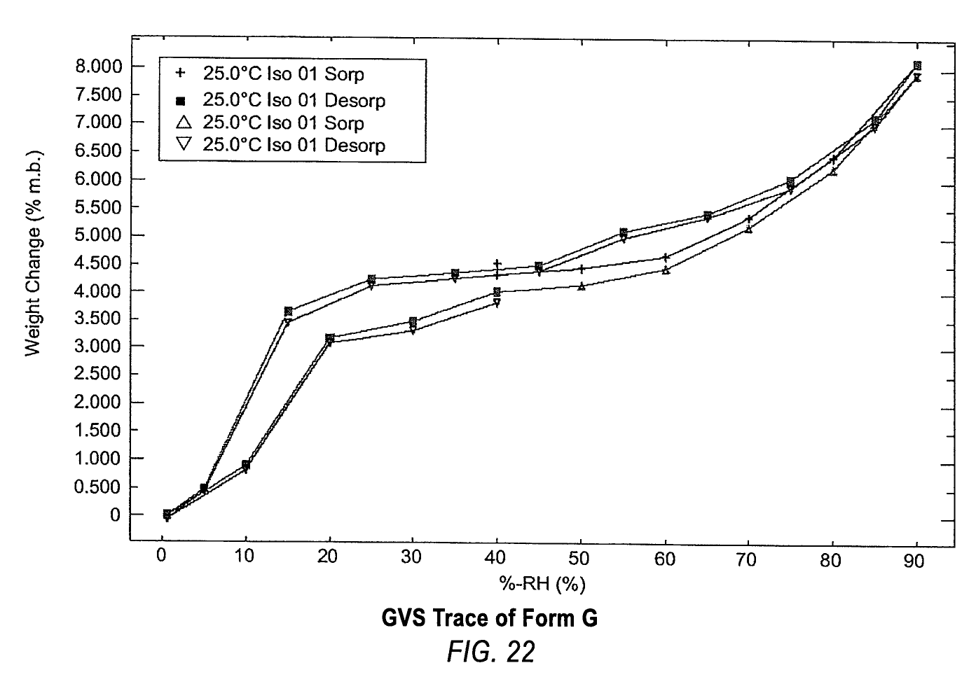

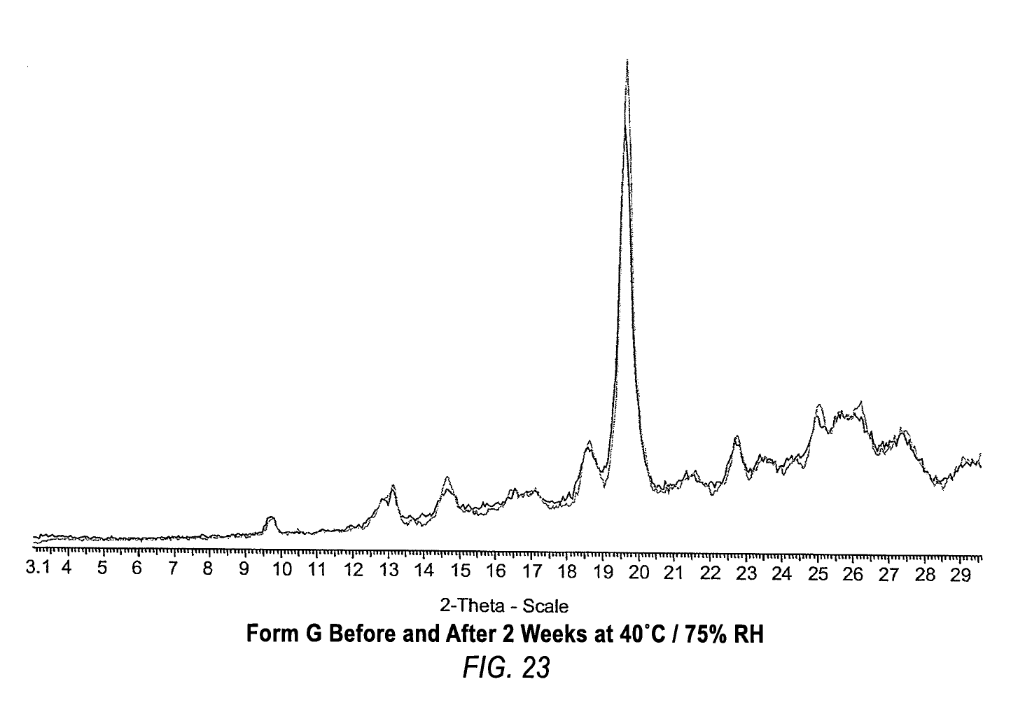

Form G was found to be highly hygroscopic, displaying a total water gain of 1% (in weight) at 10% RH and a weight gain in water of more than 8% at 90% RH as seen by GVS (FIG. 22).

Form G has been shown to convert into Form Q at a range of temperatures (e.g. from 20° C. to 50° C.), 2, 5 and 24 hours after the addition of water. See FIG. 30 for a summary of these results.

### Example IV

### Preparation of Form A

Form A was prepared by following the general procedures detailed below and Scheme I above.

Where applicable, the following HPLC method was utilized for reaction monitoring, unless otherwise indicated: a gradient of water:acetonitrile, 0.1% TFA (90:10->10:90->90:10) was run over 26 minutes at 1 mL/min and 254 nm. The method utilizes the Zorbax SB Phenyl 4.6×25 cm column, 5 μm. The term “Tᵣₑₜ” refers to the retention time, in minutes, associated with the compound.

Form A thus obtained was used in a number of solubility studies and the results are shown in Table XV below. Solubility at 60° C. was also measured in a small number of solvents. These results are shown in Table XVI.

TABLE XV
Apparent solubilities of Form A measured at room temperature

| Solvent | Solubility mg/mL |
|---|---|
| Acetonitrile | 3.8 |
| Acetone | 6.4 |
| 1-Butanol | 2.0 |
| 2-Butanone | 3.4 |
| Dimethylformamide | >100 |
| Ethanol | 5.7 |
| Ethyl Acetate | 7.2 |
| Heptane | <0.9 |
| Methanol | 5.7 |
| Methylene Chloride | <0.8 |
| 2-Propanol | <0.8 |
| 2-Propyl Acetate | 2.9 |
| Tetrahydrofuran | 1.5 |
| Water | <0.9 |

TABLE XVI
Apparent solubilities of Form A measured at 60° C.

| Solvent | Solubility mg/mL |
|---|---|
| 1-Butanol | 2.9 |
| Butyl Acetate | 1.9 |

Form A can also be obtained as a crystalline solid (obtained from the filtrate) from Form C, by applying crystallization method M described above.

Form A can be obtained by crystallization methods SE or FE described herein, using MeOH as the test solvent.

### Example IV-A

### Characterization of Form A

Form A was shown to become crystalline Form C upon heating to 100° C. Form A also become Form C described herein after one to four weeks stored at 40° C./75% RH.

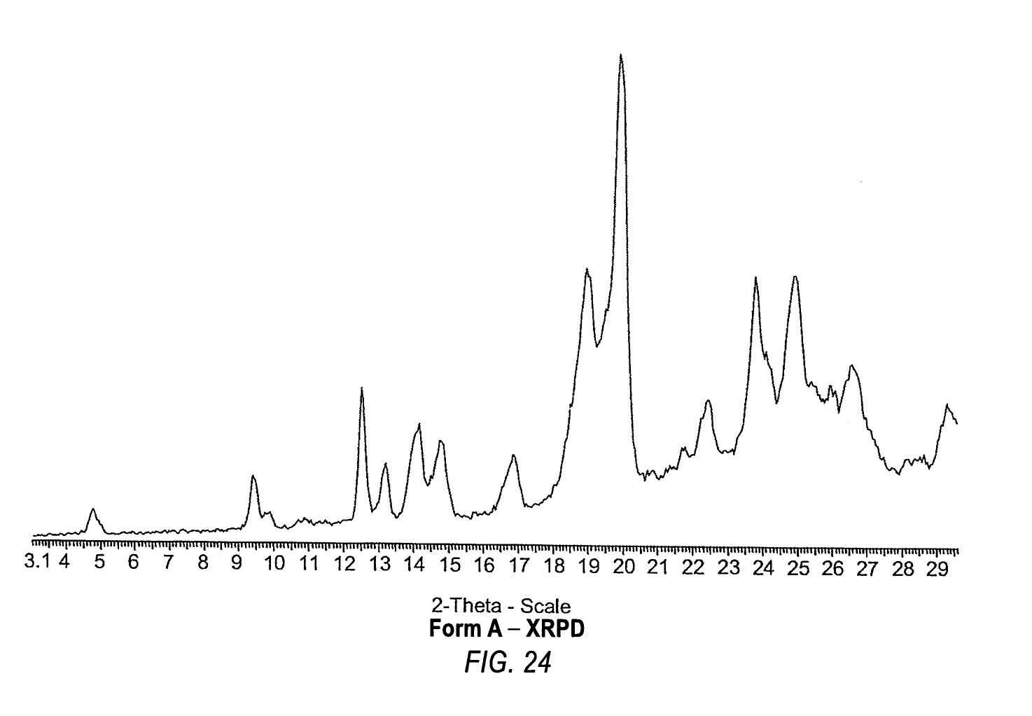

A representative XRPD pattern of Form A is provided in FIG. 24.

Form A can be characterized by a FT-IR spectrum as depicted in FIG. 27.

The DSC and TGA traces for Form A are shown in FIGS. 25 and 26, respectively. According to these, Form A can be characterized by a broad endotherm with onset 43.8° C. and peaking at 74.3° C. on DSC. Form A can be characterized by two other endotherms at 93° C. and 111° C., which are attributed to solvent loss (MeOH). Further, these coincide with a total weight loss of about 5.0% between 25° C. and 115° C. as seen by TGA.

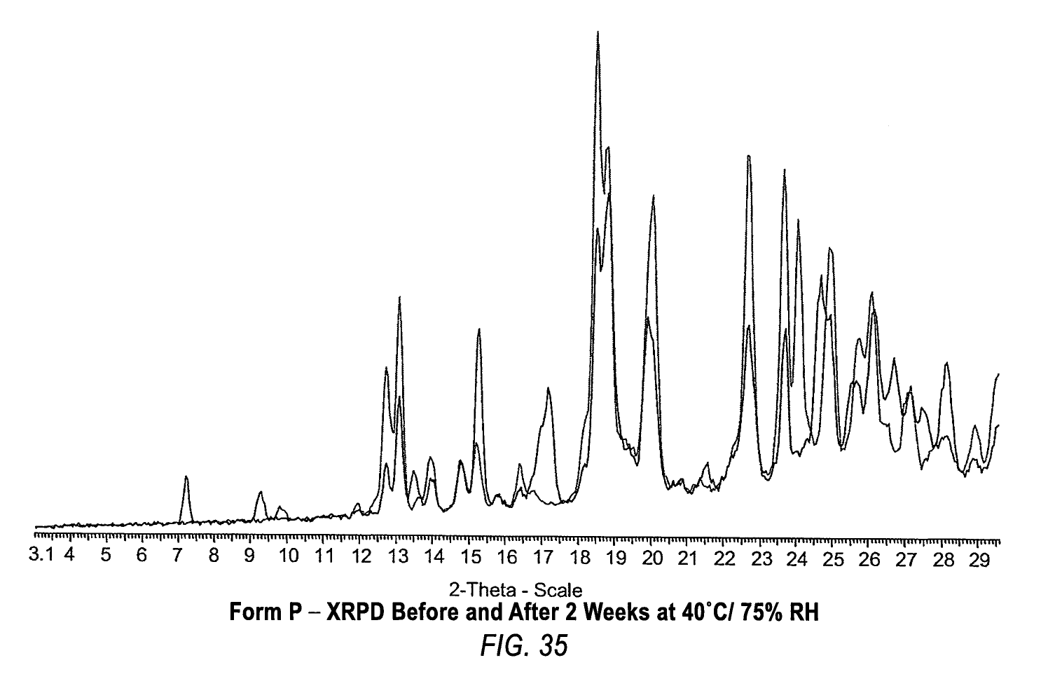

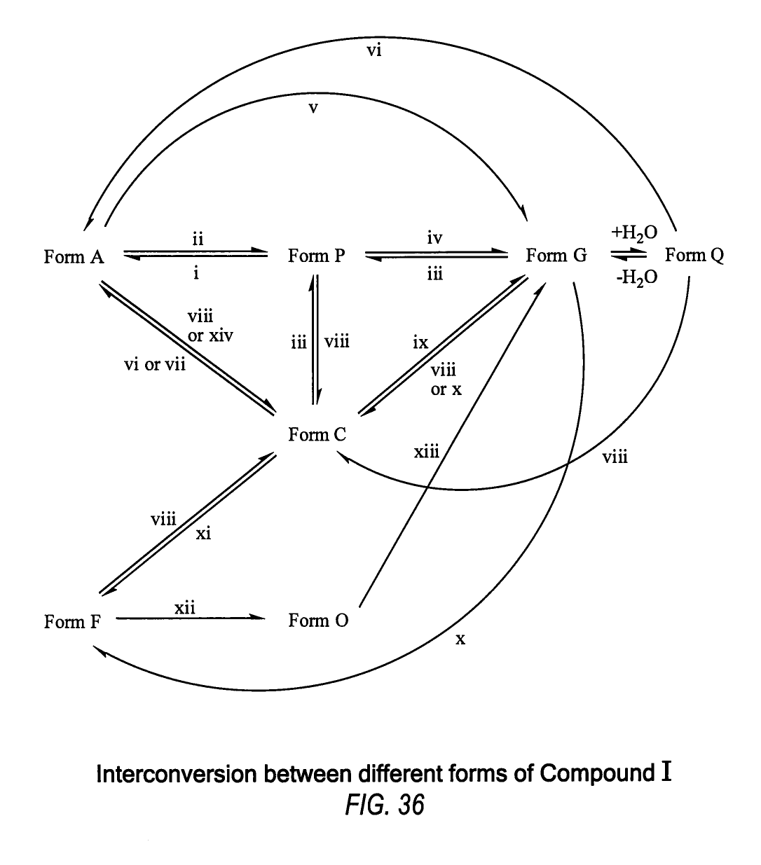

In stability studies, Form P was shown to convert to Form A upon heating to 50° C. and then to Form C upon heating to 100° C. Form A was also shown to convert to Form C upon storage at 40° C./75% RH for one week or longer. FIG. 35 (XRPD traces before and after 4 weeks stability study and comparison with Form C).

### Example V

### Preparation and Characterization of Form P

Form P is a crystalline form of Compound I and can be obtained by Form C or A.

A representative XRPD pattern of Form P is provided in FIG. 30. Form P can be characterized by the representative TGA and DSC traces provided in FIGS. 32 and 33, respectively.

Form P has been shown to convert to Form G after about 2 weeks of storage at 4° C.

### Example VI

### Preparation and Characterization of Form Q

Form Q is a crystalline form of Compound I and its characterized as a 1:1 hydrate of Form G. Form Q can be obtained by adding water to Form G and storing it at room temperature.

A representative XRPD pattern of Form Q is provided in FIG. 29.

### Comparison Studies

### Example IX

### Interconversion Studies

Interconversion studies between the different forms of Compound I described herein were carried out using the general procedure outlined here:

Interconversion studies were conducted in EtOAc, MeOH and water with materials giving XRPD patterns A, P, C and F. Attempts were made to monitor the presence of the different forms of Compound I in the methanol and ethyl acetate interconversion slurries using Raman analysis and XRPD. The presence of the solvents was dominant in the Raman spectra of the slurries. The slurries exhibited crystalline XRPD patterns, however, the patterns were not directly comparable with previous patterns possibly due to shifting of the slurry during the XRPD analysis. The material from a methanol slurry exhibited additional peaks. After slurrying for 10 days, pattern A was obtained from MeOH. Pattern C was obtained from all samples slurried in EtOAc or water. Form C appears to be the most stable unsolvated form at ambient conditions. Interconversion data can be found in Tables XVII.

TABLE XVII
Interconversion Studies of
Compound I, Patterns A, C and F

| Solvent | Resulted pattern |
|---|---|
| EtOAc | Crystalline |
| | Pattern C |
| MeOH | Crystalline |
| | Pattern A |
| H₂O | Pattern C |

### Example 10

### Relative Stability Studies

### Study I: Relative Stability of Forms C and G

The following procedure was employed:

1:1 mixtures of Form C and form G of Compound I were slurried in 3:1 water:ethanol at a range of temperatures to determine the relative stability at different temperatures. 5 mg of Form C was mixed with 5 mg of Form G in a glass vial. 1.0 ml of 3:1 water:ethanol was added and the resulting slurry was stirred at 5° C. for 10 days. The resulting solid was isolated by filtration and analysed by XRPD (see FIG. 17).

The above procedure was repeated at 25, 50 and 80° C. Results are recorded in Table XVIII.

TABLE XVIII
Slurrying of Forms C and G at Various Temperatures

| Temperature (° C.) | XRPD |
|---|---|
| 5 | Mix of form C and form G |
| 25 | Mix of form C and form G |
| 50 | Form C |

The results indicate that Form C is more stable than Form G at a temperature of 50° C. or above. At 5 and 25° C., both forms were present after 10 days of slurrying, suggesting that the difference in stability between Forms C and G at these temperatures is small.

### Study II. Relative Stability of Forms C and F

The following procedure was used:

1:1 mixtures of Form C and Form F were slurried in 3:1 water:ethanol at a range of temperatures to determine the relative stability at different temperatures. The form remaining after the slurrying should be the more stable form. Ethanol was used in addition to water in order to increase the amount of Compound I in solution and so increase the rate of conversion between forms. Ethanol was chosen as no ethanol solvate of Compound I was known. 10 mg of Form C was mixed with 10 mg of Form F in a glass vial. 2.0 ml of 3:1 water:ethanol was added and the resulting slurry was stirred at 5° C. for 24 hours. Solid was isolated by filtration and analysed by XRPD.

The above procedure was repeated at 25, 50 and 80° C. Results are recorded in Table XIX.

TABLE XIX
Slurrying of Forms C and F at Various Temperatures

| Temperature (° C.) | XRPD |
|---|---|
| 5 | Form C with some form G |
| 25 | Form C with some form G |
| 50 | Form C |

The results indicate that Form C is more stable than Form F at 50 and 80° C. At 5 and 25° C., Form F was observed to convert to Form G. The duration of slurrying at and 25° C. was not sufficient to determine whether Form G or C was the most stable at these temperatures.

### Study III. Relative Stability of Forms F and G

The following general procedure was employed:

1:1 mixtures of Form F and Form G were slurried in 3:1 water:ethanol at a range of temperatures to determine the relative stability at different temperatures. 10 mg of Form F was mixed with 10 mg of Form G in a glass vial. 2.0 ml of 3:1 water:ethanol was added and the resulting slurry was stirred at 5° C. for 24 hours. The resulting solid was isolated by filtration and analysed by XRPD. The above procedure was repeated at 25, 50 and 70° C. 70° C. was used instead of 80° C. in an attempt to reduce degradation. Results are recorded in Table XX.

TABLE XX
Slurrying of Forms F and G at Various Temperatures

| Temperature (° C.) | XRPD |
|---|---|
| 5 | Form G |
| 25 | Form G with some form C |
| 50 | Form C with some form G |

The results indicate that Form G is more stable than Form F at 5° C. At all other temperatures, there was some conversion to Form C. Form F was not recovered after slurrying at any of the four temperatures.

1. Crystalline 2-(2,4-difluorophenyl)-6-(1-(2,6-difluorophenyl)ureido)nicotinamide.

2. The crystalline 2-(2,4-difluorophenyl)-6-(1-(2,6-difluorophenyl)ureido)nicotinamide of claim 1, characterized by peaks at least at 7.4 degrees, at 9.5 degrees, at 15.5 degrees, at 17.2 degrees, and at 24.8 degrees in an X-Ray powder diffraction pattern.

3. The crystalline 2-(2,4-difluorophenyl)-6-(1-(2,6-difluorophenyl)ureido)nicotinamide of claim 2, characterized by a peak at 13.7 degrees, at 14.1 degrees, at 19.2 degrees, at 22.9 degrees, at 26.3 degrees, at 26.9 degrees, at 27.7 degrees, at 28.3 degrees in an X-Ray powder diffraction pattern.

4. The crystalline 2-(2,4-difluorophenyl)-6-(1-(2,6-difluorophenyl)ureido)nicotinamide of claim 1, characterized by an X-Ray powder diffraction pattern substantially similar to FIG. 1.

5. The crystalline 2-(2,4-difluorophenyl)-6-(1-(2,6-difluorophenyl)ureido)nicotinamide of claim 1, characterized by a space group C_{c} as revealed by single crystal X-Ray crystallography.

6. The crystalline 2-(2,4-difluorophenyl)-6-(1-(2,6-difluorophenyl)ureido)nicotinamide of claim 5 having the following unit cell dimensions, as determined by single crystal X-Ray crystallography:
a=10.92 Å, b=24.20 Å, c=7.01 Å, α=90°, β=111.07°, and γ=90°.

7. The crystalline 2-(2,4-difluorophenyl)-6-(1-(2,6-difluorophenyl)ureido)nicotinamide of claim 1 displaying a ¹H NMR spectrum substantially similar to that depicted in FIG. 2.

8. The crystalline 2-(2,4-difluorophenyl)-6-(1-(2,6-difluorophenyl)ureido)nicotinamide of claim 1 displaying a FT-IR spectrum substantially similar to that depicted in FIG. 3.

9. The crystalline 2-(2,4-difluorophenyl)-6-(1-(2,6-difluorophenyl)ureido)nicotinamide of claim 1, characterized by a solubility in water of at least 0.02 mg/mL at 25° C.

10. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide remains in substantially the same physical form for at least two weeks at 40° C./75% relative humidity.

11. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide displays a negligible weight gain up to 60% relative humidity and a total weight gain of 0.15% from 0 to 90% relative humidity at T=25° C.

12. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide remains chemically stable for at least 2 weeks at 40° C./75% relative humidity.

13. A method of making crystalline 2-(2,4-difluorophenyl)-6-(1-(2,6-difluorophenyl)ureido)nicotinamide of claim 1 from crystalline Form A, the method comprising the steps of:
ii) slurrying methanol solvate Form A in 20 volumes of a 1:3 methanol:water mixture for 24 hours (a kinetically controlled step that produces Form C and Form Q/G, described above), and
iii) slurrying the resulting mixture in a 1:1 methanol:water mixture to suppress formation of Form Q/G and favor thermodynamically more stable Form C.

14. The crystalline 2-(2,4-difluorophenyl)-6-(1-(2,6-difluorophenyl)ureido)nicotinamide of claim 1, characterized by a peak at 14.0 degrees, at 15.6 degrees, at 17.3 degrees, at 19.1 degrees, at 20.4 degrees, at 23.1 degrees, and at 24.9 degrees in an X-Ray powder diffraction pattern.

15. The crystalline 2-(2,4-difluorophenyl)-6-(1-(2,6-difluorophenyl)ureido)nicotinamide of claim 1, characterized by an X-Ray powder diffraction pattern substantially similar to FIG. 11.

16. The crystalline 2-(2,4-difluorophenyl)-6-(1-(2,6-difluorophenyl)ureido)nicotinamide of claim 1 displaying a ¹H NMR spectrum substantially similar to that depicted in FIG. 12.

17. The crystalline 2-(2,4-difluorophenyl)-6-(1-(2,6-difluorophenyl)ureido)nicotinamide of claim 1 displaying a FT-IR spectrum substantially similar to that depicted in FIG. 13.

18. The crystalline 2-(2,4-difluorophenyl)-6-(1-(2,6-difluorophenyl)ureido)nicotinamide of claim 1 characterized by a solubility of at least 0.021 mg/mL at 25° C.

19. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide remains in substantially the same physical form for at least 2 weeks at 40° C./75% relative humidity.

20. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide displays a total weight gain in water of 1% at 40% relative humidity and a maximum of 1.1% at 90% relative humidity.

21. A method of making crystalline 2-(2,4-difluorophenyl)-6-(1-(2,6-difluorophenyl)ureido)nicotinamide of claims 1 from crystalline Form C, the method comprising the steps of:
iv) preparing an ethyl acetate slurry of Form C,
v) precipitating it with cold hexanes for 2 h, and
vi) filtering and drying the resulting solid to furnish Compound I, Form F.

22. The crystalline 2-(2,4-difluorophenyl)-6-(1-(2,6-difluorophenyl)ureido)nicotinamide of claim 1, characterized by a peak at 9.9 degrees, at 14.8 degrees, at 17.3 degrees, at 18.8 degrees, at 19.8 degrees, at 21.7 degrees, at 22.7 degrees, at 23.6 degrees, and at 27.7 degrees in an X-Ray powder diffraction pattern.

23. The crystalline 2-(2,4-difluorophenyl)-6-(1-(2,6-difluorophenyl)ureido)nicotinamide of claim 1, characterized by an X-Ray powder diffraction pattern substantially similar to FIG. 17.

24. The crystalline 2-(2,4-difluorophenyl)-6-(1-(2,6-difluorophenyl)ureido)nicotinamide of claim 1 displaying a ¹H NMR spectrum substantially similar to that depicted in FIG. 18.

25. The crystalline 2-(2,4-difluorophenyl)-6-(1-(2,6-difluorophenyl)ureido)nicotinamide of claim 1 displaying a FT-IR spectrum substantially similar to that depicted in FIG. 19.

26. The crystalline 2-(2,4-difluorophenyl)-6-(1-(2,6-difluorophenyl)ureido)nicotinamide of claim 1, characterized by a solubility of at least 0.020 mg/mL at 25° C.

27. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide remains in substantially the same physical Form for at least two weeks at 40° C./75% relative humidity.

28. The crystalline 2-(2,4-difluorophenyl)-6-(1-(2,6-difluorophenyl)ureido)nicotinamide of claim 1, wherein the crystalline 2-(2,4-difluorophenyl)-6-(1-(2,6-difluorophenyl)ureido)nicotinamide displays a total weight gain in water of 1% at 10% relative humidity and a weight gain in water over 8% at 90% relative humidity.

29. A method of making crystalline 2-(2,4-difluorophenyl)-6-(1-(2,6-difluorophenyl)ureido)nicotinamide of claim 1 from crystalline Form C, the method comprising the steps of:
vii) preparing an ethyl acetate slurry of Form C,
viii) precipitating it with cold hexanes for 24 h, and
iii) filtering and drying the resulting solid to furnish Compound I, Form G.

30. A method of making crystalline 2-(2,4-difluorophenyl)-6-(1-(2,6-difluorophenyl)ureido)nicotinamide of claim 1 from hydrate Form Q by dehydration at room temperature.

31. The crystalline 2-(2,4-difluorophenyl)-6-(1-(2,6-difluorophenyl)ureido)nicotinamide of claim 1, characterized by a peak at 13.4 degrees, at 14.2 degrees, at 15.1 degrees, at 17.1 degrees, at 19.1 degrees, at 20.1 degrees, and at 25.0 degrees in an X-Ray powder diffraction pattern.

32. The crystalline 2-(2,4-difluorophenyl)-6-(1-(2,6-difluorophenyl)ureido)nicotinamide of claim 1, characterized by an X-Ray powder diffraction pattern substantially similar to that shown in FIG. 24.

33. The crystalline 2-(2,4-difluorophenyl)-6-(1-(2,6-difluorophenyl)ureido)nicotinamide of claim 1, characterized by a solubility of 0.016-0.018 mg/mL at 25° C.

34. The crystalline 2-(2,4-difluorophenyl)-6-(1-(2,6-difluorophenyl)ureido)nicotinamide of claim 1 displaying a FT-IR spectrum substantially similar that depicted in FIG. 27.

35. The crystalline 2-(2,4-difluorophenyl)-6-(1-(2,6-difluorophenyl)ureido)nicotinamide of claim 1, characterized by a peak at 12.9 degrees, at 13.3 degrees, at 18.9 degrees, at 20.2 degrees, at 20.4 degrees, at 25.2 degrees, and at 25.8 degrees in an X-Ray powder diffraction pattern.

36. The crystalline 2-(2,4-difluorophenyl)-6-(1-(2,6-difluorophenyl)ureido)nicotinamide of claim 1, characterized by an X-Ray powder diffraction pattern substantially similar to FIG. 30.

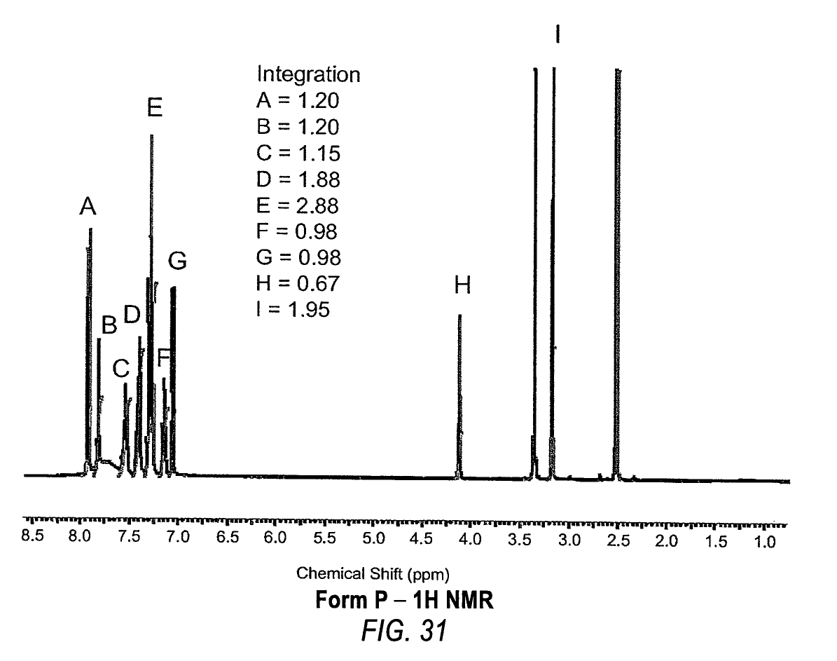

37. The crystalline 2-(2,4-difluorophenyl)-6-(1-(2,6-difluorophenyl)ureido)nicotinamide of claim 1 displaying a ¹H NMR spectrum substantially similar to that depicted in FIG. 31.

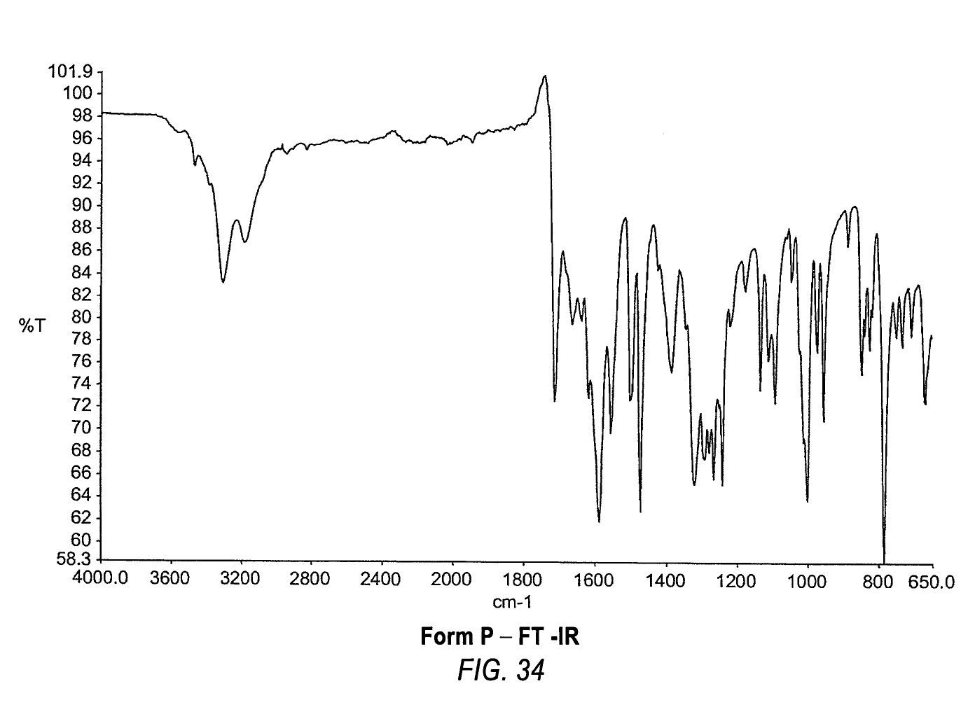

38. The crystalline 2-(2,4-difluorophenyl)-6-(1-(2,6-difluorophenyl)ureido)nicotinamide of claim 1 displaying a FT-IR spectrum substantially similar to that depicted in FIG. 34.

39. The crystalline 2-(2,4-difluorophenyl)-6-(1-(2,6-difluorophenyl)ureido)nicotinamide of claim 1, characterized by conversions to Form C upon storage at 40° C./75% relative humidity for 72 h.

40. The crystalline 2-(2,4-difluorophenyl)-6-(1-(2,6-difluorophenyl)ureido)nicotinamide of claim 1, characterized by conversions to Form C upon storage at 4° C. for 72 h.

41. The crystalline 2-(2,4-difluorophenyl)-6-(1-(2,6-difluorophenyl)ureido)nicotinamide of claim 1, characterized by a peak at 10.1 degrees, at 12.8 degrees, at 14.2 degrees, at 15.8 degrees, at 16.7 degrees, at 18.8 degrees, at 19.9 degrees, at 20.2 degrees, at 22.9 degrees, at 23.8 degrees, at 24.9 degrees, and at 29.8 degrees in an X-Ray powder diffraction pattern.

42. A pharmaceutical composition comprising crystalline 2-(2,4-difluorophenyl)-6-(1-(2,6-difluorophenyl)ureido)nicotinamide of claim 2 and a pharmaceutically acceptable carrier.
